(19) United States
(12) Patent Application Publication
(10) Pub. No.: US 2026/0265732 A1
(43) Pub. Date: Sep. 10, 2026

(54) SPATIALLY ENCAPSULATED DNA ENCODED LIBRARIES AND METHODS OF SCREENING SAME

(71) Applicant: OM THERAPEUTICS, INC., San Diego, CA (US)

(72) Inventors: Mohamed BOUZAFFOUR, San Diego, CA (US); Devon CAYER, San Diego, CA (US)

(21) Appl. No.: 19/666,158

(22) Filed: May 1, 2026

Related U.S. Application Data
(63) Continuation of application No. PCT/US2024/054475, filed on Nov. 4, 2024.
(60) Provisional application No. 63/547,131, filed on Nov. 2, 2023.

(51) Int. Cl. C12N 15/10; C12Q 1/6806; C12Q 1/6844; C12Q 1/6869; C40B 40/08; C40B 40/10; G16B 15/30

(52) U.S. Cl. CPC C12N 15/1093; C12N 15/1089; C12Q 1/6806; C12Q 1/6844; C12Q 1/6869; C40B 40/08; C40B 40/10; G16B 15/30

(57) ABSTRACT

Provided herein are nucleic acid encoded compositions, nucleic acid encoded library compositions, and methods of making and using the nucleic acid encoded compositions and nucleic acid encoded library compositions.

### CROSS-REFERENCE TO RELATED APPLICATIONS

The present application is a Continuation of International Application No. PCT/US2024/054475, filed on Nov. 4, 2024, which claims priority to U.S. Provisional Application No. 63/547,131, filed on Nov. 2, 2023. The contents of the above-referenced application are herein expressly incorporated by reference herein in their entirety, including any drawings.

### SUMMARY

In an aspect, provided herein are, inter alia, are a method of screening for one or more DNA encoded compounds that bind to a target polypeptide is provided, the method comprising: (i) contacting a plurality of DNA encoded compounds with a target polypeptide bound to a microparticle under conditions that allow for binding of the plurality of DNA encoded compounds to the target polypeptide, wherein the microparticle is bound to a nucleic acid multifunctional sequence, wherein said nucleic acid multifunctional sequence comprises: a nucleic acid polypeptide identifier sequence; and a first hybridization sequence; wherein each DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, (ii) removing DNA encoded compounds not binding to the polypeptide; (iii) compartmentalizing each microparticle; (iv) amplifying the DNA compound sequence to produce a complement of the DNA compound sequence, wherein the complement of the DNA compound sequence comprises a complement of the second hybridization sequence; (v) hybridizing the complement of the second hybridization sequence to the first hybridization sequence; and (vi) elongating the nucleic acid multifunctional sequence with a polymerase to produce a nucleic acid identification sequence comprising the nucleic acid compound identifier sequence and the nucleic acid polypeptide identifier sequence, thereby identifying the one or more DNA encoded compounds that bind to a target polypeptide.

In an aspect, a method of screening for one or more DNA encoded compounds that bind to a target polypeptide is provided, the method comprising: (i) contacting a plurality of DNA encoded compounds with a plurality of target polypeptides bound to a microparticle under conditions that allow for binding of the plurality of DNA encoded compounds to the polypeptides, wherein the plurality of target polypeptides bound to a microparticle comprises a nucleic acid multifunctional sequence bound to said microparticle, wherein said nucleic acid multifunctional sequence comprises: a nucleic acid polypeptide identifier sequence; and a first hybridization sequence; wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein the complement of the second hybridization sequence is capable of hybridizing to said first hybridization sequence, (ii) removing DNA encoded compounds not binding to a target polypeptide; (iii) compartmentalizing each microparticle; and (iv) amplifying the DNA compound sequence to produce a complement of the DNA compound sequence, wherein the complement of the DNA compound sequence comprises a complement of the second hybridization sequence; (v) hybridizing the complement of the second hybridization sequence to the first hybridization sequence; and (vi) elongating the nucleic acid multifunctional sequence with a polymerase to produce a nucleic acid identification sequence comprising the nucleic acid compound identifier sequence and the nucleic acid polypeptide identifier, thereby identifying the one or more DNA encoded compounds that bind to a target polypeptide.

In some embodiments, the method further comprises de-coding the compound from the corresponding nucleic acid compound identifier sequence.

In aspects, a method of screening for one or more DNA encoded compounds that bind to a target polypeptide is provided, the method comprising: contacting a plurality of DNA encoded compounds with a target polypeptide bound to a microparticle under conditions that allow the DNA encoded compounds to bind the target polypeptide, wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, wherein the microparticle comprises a nucleic acid multifunctional sequence bound to said microparticle, wherein said nucleic acid multifunctional sequence comprises: a nucleic acid polypeptide identifier sequence; and a first hybridization sequence, removing DNA encoded compounds not binding to the target polypeptide; compartmentalizing each microparticle; and linking each target polypeptide bound to a DNA encoded compound to the DNA encoded compound by hybridizing the first and second hybridization sequences, thereby identifying the one or more DNA encoded compounds that specifically bind to a target polypeptide by de-coding from corresponding nucleic acid compound identifier sequence.

In aspects, a method of screening for one or more DNA encoded compounds that bind to a target polypeptide is provided, the method comprising: contacting a plurality of DNA encoded compounds with a plurality of target polypeptides bound to a microparticle under conditions that allow the DNA encoded compounds to bind the target polypeptides, wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, wherein the plurality of target polypeptides bound to a microparticle comprises a nucleic acid multifunctional sequence bound to said microparticle, wherein said nucleic acid multifunctional sequence comprises: a nucleic acid polypeptide identifier sequence; and a first hybridization sequence, removing DNA encoded compounds not binding to a target polypeptide; compartmentalizing each microparticle; and linking each target polypeptide bound to a DNA encoded compound to the DNA encoded compound by hybridizing the first and second hybridization sequences, thereby identifying the one or more DNA encoded compounds that bind to a target polypeptide by de-coding from corresponding nucleic acid compound identifier sequence.

In embodiments, the method further comprises amplifying the DNA compound sequence. In embodiments, the method further comprises amplifying the DNA compound sequence hybridized to the nucleic acid multifunctional sequence, wherein the amplification forms a nucleic acid identification sequence. The amplification reaction, such as PCR, may be performed using any suitable method known in the art.

In embodiments, the method further comprises identifying the one or more target polypeptides that bind to a DNA encoded compound by de-coding from corresponding nucleic acid polypeptide identifier nucleic acid sequences. In embodiments, the method further comprises amplifying a nucleic acid identification sequence comprising the nucleic acid polypeptide identifier sequence, the first hybridization sequence, and the nucleic acid compound identifier sequence. In embodiments, the method further comprises sequencing the nucleic acid identification sequence. In embodiments, the method further comprises identifying and determining unique identifier sequencing events. In embodiments, the method further comprises determining the number of target polypeptide binding events for each unique nucleic acid compound identifier sequence. In embodiments, the method further comprises determining a target polypeptide binding frequency for each DNA encoded compound. In embodiments, the method further comprises summing all target polypeptide binding events across two or more microparticles comprising the same target polypeptide. In embodiments, the method further comprises determining a distribution of target polypeptide binding events. In embodiments, the method further comprises repeating the method steps. In embodiments, the method further comprises determining a distribution of target polypeptide binding events over two or more repeats.

In an aspect, a computer-implemented method for identifying a compound having an affinity with a target polypeptide, the method comprising querying a machine learning engine for a proposed compound having an affinity with the target polypeptide, wherein the machine learning engine was trained using target polypeptide binding events of the disclosure; and receiving the compound from the machine learning engine.

In aspect, provided herein is a composition comprising a microparticle; a target molecule bound to said microparticle; and a nucleic acid multifunctional sequence bound to said microparticle, wherein said nucleic acid multifunctional sequence comprises: a nucleic acid target molecule identifier sequence; and a first hybridization sequence.

In aspect, provided herein is a composition comprising a microparticle; a target molecule bound to said microparticle; a nucleic acid multifunctional sequence bound to said microparticle, wherein said nucleic acid multifunctional sequence comprises: a nucleic acid target molecule identifier sequence; and a first hybridization sequence; and a DNA encoded compound bound to said target molecule, said DNA encoded compound comprising a compound bound to a DNA compound sequence, said DNA compound sequence comprising: a nucleic acid compound identifier sequence; and a second hybridization sequence wherein a complement of the second hybridization sequence is capable of hybridizing to said first hybridization sequence.

In aspect, provided herein is a composition comprising a microparticle; a target molecule bound to said microparticle; a nucleic acid multifunctional sequence bound to said microparticle, wherein said nucleic acid multifunctional sequence comprises: a nucleic acid target molecule identifier sequence; a first hybridization sequence; and a DNA encoded compound bound to said nucleic acid multifunctional sequence, said DNA encoded compound comprising a compound bound to a DNA compound sequence, said DNA compound sequence comprising: a nucleic acid compound identifier sequence; and a complement of a second hybridization sequence, wherein the complement of the second hybridization sequence and the first hybridization sequence are bound to each other.

In some embodiments, the target molecule is a target polypeptide.

In aspect, provided herein is a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; and a first hybridization sequence; and a DNA encoded compound bound to said target polypeptide, said DNA encoded compound comprising a compound bound to a DNA compound sequence, said DNA compound sequence comprising: a nucleic acid compound identifier sequence; and a second hybridization sequence wherein a complement of the second hybridization sequence is capable of hybridizing to said first hybridization sequence.

In aspect, provided herein is a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; a first hybridization sequence; and a DNA encoded compound bound to said nucleic acid multifunctional sequence, said DNA encoded compound comprising a compound bound to a DNA compound sequence, said DNA compound sequence comprising: a nucleic acid compound identifier sequence; and a complement of a second hybridization sequence, wherein the complement of the second hybridization sequence and the first hybridization sequence are bound to each other.

In an aspect, the nucleic acid multifunctional sequence further comprises a target polypeptide encoding nucleic acid sequence.

In embodiments, the composition further comprises a nucleic acid identification sequence comprising the nucleic acid target molecule identifier sequence or nucleic acid polypeptide identifier sequence, the first hybridization sequence, and the nucleic acid compound identifier sequence.

In embodiments, the microparticle is a hydrogel. In embodiments, the longest dimension of the microparticle is at least 10 μM. In embodiments, the longest dimension of the microparticle is at most 500 M. In embodiments, the microparticle is a bead.

In embodiments, the target polypeptide further comprises a protein tag. In embodiments, the protein tag is selected from the group of Polyhistidine (His-tag), Glutathione S-transferase (GST), FLAG tag, SPY tag, calmodulin binding domain (CBD), chitin binding domain (CBD), choline-binding domain (CBD), albumin-binding protein (ABP), Bacteriophage T7 epitope (T7-tag), bacteriophage V5 epitope (V5-tag), biotin-carboxy carrier protein (BCCP), bluetongue virus tag (B-tag), chloramphenicol acetyl transferase (CAT), cellulose binding domain (CBP), E2 epitope, galactose-binding protein (GBP), green fluorescent protein (GFP), Glu-Glu (EE-tag), human influenza hemagglutinin (HA), HaloTag, maltose-binding protein (MBP), streptavadin-binding peptide (SBP), Myc epitope tag, strep-tag, and biotin.

In embodiments, the target polypeptide is bound non-covalently to the microparticle. In embodiments, the target polypeptide is bound covalently to the microparticle. In embodiments, the target polypeptide is bound to the microparticle by a target polypeptide binding moiety. In embodiments, the target polypeptide binding moiety is selected from the group of a divalent metal, glutathione, anti-FLAG antibody or fragment thereof, calmodulin, chitin, choline, anti-E2 antibody or a fragment thereof, cellulose, albumin, streptavidin, anti-bluetongue virus antibody or a fragment thereof, galactose, anti-GFP antibody or a fragment thereof, biotin, target polypeptide specific antibody, and maltose.

In embodiments, the target polypeptide binding moiety further comprises a linker. In embodiments, the linker is a polypeptide linker, a chemical linker or a nucleic acid linker. In embodiments, where the linker of chemical linker (e.g., a biomolecule such as a polypeptide, polypeptide binding moiety, or nucleic acid) is covalently bound to a microparticle as described herein, the covalent attachment is achieved using bioconjugate chemistry as described herein. For example, the linker may be covalently bound to the microparticle through a bioconjugate as described herein.

In embodiments, the composition is encapsulated. In embodiments, the composition is partitioned into a compartment. In an aspect, the composition is encapsulated in the same compartment. In embodiments, the compartment is an aqueous droplet within emulsion. In embodiments, the emulsion is an oil in water emulsion.

In embodiments, wherein the compound is selected from the group of a small molecule, a macromolecule, a peptide, a polypeptide, a protein, a protein complex, a nucleic acid, a lipid, a carbohydrate, a glycan, and a cell. In embodiments, the compound is a biomolecule.

In embodiments, the compound is a protein complex. In embodiments, the compound is a neoantigen peptide. In embodiments, the compound is a RNA, a DNA, or a mixture of RNA or DNA. In embodiments, the compound is an aptamer. In embodiments, the compound is a peptide nucleic acid (PNA) or other genetic polymers.

In embodiments, the nucleic acid multifunctional sequence is bound to the microparticle by a nucleic acid linker. In embodiments, the nucleic acid multifunctional sequence further comprises a unique identifier. In embodiments, the nucleic acid multifunctional sequence is a DNA, RNA, or a mixture of DNA and RNA, or alternate genetic polymers. In embodiments, the nucleic acid multifunctional sequence is a single stranded nucleic acid. In embodiments, the nucleic acid multifunctional sequence is a double stranded nucleic acid and the 5′ and 3′ ends each comprise a hairpin of unpaired bases and the hairpin connects the 5′ end of one strand to the 3′ end of the other strand. In embodiments, the hairpins are at least 10 bases long.

In embodiments, the nucleic acid multifunctional sequence further comprises a first amplification sequence. In embodiments, the DNA compound sequence further comprises a second amplification sequence.

In embodiments, the polypeptide encoding nucleic acid sequence encodes all or a part of the target polypeptide.

In embodiments, about 10⁵ to 10⁶ of said nucleic acid multifunctional sequences are attached to the microparticle. In embodiments, the nucleic acid multifunctional sequences are the same. In embodiments, about 10⁶ to 107 of said target polypeptides are attached to the microparticle. In embodiments, the target polypeptides are the same.

In an aspect, a method of making a composition of the disclosure is provided, the method comprising combining the microparticle, a target molecule binding moiety, the target molecule, the nucleic acid multifunctional sequence and the DNA encoded compound under conditions sufficient for (i) attaching the target molecule binding moiety and the nucleic acid multifunctional sequence to the microparticle, and (ii) binding the target molecule to the target molecule binding moiety.

In an aspect, a method of making a composition of the disclosure is provided, the method comprising combining the microparticle, a target molecule binding moiety, the target molecule, the nucleic acid multifunctional sequence and the DNA encoded compound under conditions sufficient for (i) attaching the target molecule binding moiety and the nucleic acid multifunctional sequence to the microparticle, (ii) binding the target molecule to the target molecule binding moiety; and (iii) binding the DNA encoded compound to the target molecule.

In embodiments, step (i) comprises covalently conjugating the multifunctional sequence to the microparticle. In embodiments, step (i) comprises covalently or non-covalently conjugating a target molecule binding moiety to the microparticle.

In an aspect, a method of making a composition of the disclosure is provided, the method comprising combining the microparticle, a target polypeptide binding moiety, the polypeptide, the nucleic acid multifunctional sequence and the DNA encoded compound under conditions sufficient for (i) attaching the target polypeptide binding moiety and the nucleic acid multifunctional sequence to the microparticle, (ii) binding the target polypeptide to the target polypeptide binding moiety; and (iii) binding the DNA encoded compound to the target polypeptide.

In embodiments, step (i) comprises covalently conjugating the multifunctional sequence to the microparticle. In embodiments, step (i) comprises covalently or non-covalently conjugating a target polypeptide binding moiety to the microparticle.

In embodiments, the conjugation is a chemical conjugation. In embodiments, the microparticle comprises one or more functional reactive groups. In embodiments, the functional reactive group is a bioconjugate reactive group. In embodiments, the functional reactive group is an amine group or a carboxy group. In embodiments, the functional reactive group is functionalized with a dibenzylcyclooctyne (DBCO). In embodiments, the method further comprises, reacting the DBCO with a nucleic acid multifunctional sequence azide. In embodiments, the method further comprises, reacting the DBCO with a target polypeptide binding moiety azide.

In embodiments, the method further comprises, encapsulating the composition.

In embodiments, the method further comprises, producing the target polypeptide in situ by transcribing and translating the target polypeptide encoding nucleic acid sequence.

In aspect, provided herein is a library, wherein the library comprises a plurality of compositions of the disclosure, or a plurality of compositions produced by the method of the disclosure.

In some embodiments, the library comprises a plurality of target polypeptides. In some embodiments, the target polypeptides are the same target polypeptide. In some embodiments, target polypeptides are different target polypeptides.

In some embodiments, the library comprises a plurality of DNA encoded compounds. In some embodiments, DNA encoded compounds are the same compound. In some embodiments, in the DNA encoded compounds are different compounds.

In some embodiments, the library is in an emulsion. In some embodiments, each member of the library is encapsulated. In some embodiments, each member of the library is partitioned into a different compartment. In some embodiments, each compartment is an aqueous droplet in an emulsion. In some embodiments, the emulsion is an oil in water emulsion. In some embodiments, each compartment comprises not more than a single microparticle. In some embodiments, the library is in a vessel. In some embodiments, each member of the library is in a vessel.

In some embodiments, the target polypeptide is derived from a transcriptome.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a picture of microparticles after functionalization of with Click Chemistry Groups. Microparticles were functionalized with two orthogonal click chemistry groups. The efficiency of functionalization was confirmed by conjugating a Cyanine 5 dye via click chemistry, demonstrating successful labeling of the microparticles.

FIG. 2 shows pictures microparticles after functionalization. Top panel shows a visualization of C-terminal GST-tagged GFP protein immobilized on microparticles coated with a polyclonal anti-GST antibody. Bottom panel shows a visualization of a Cy5-labeled DNA probe hybridized to an oligonucleotide grafted to the surface of the microparticles.

FIG. 3 shows brightfield and Cy5 fluorescence images for Quality Control of Particle Barcoding Ligation by Fluorescent Imaging. Polymer particles subjected to either a complete ligation mix (positive control) or a ligation mix without the splint (negative control) were hybridized with a fluorescently labeled DNA probe complementary to the module being ligated. A fluorescent signal is observed only in the positive control, confirming successful ligation of oligonucleotide barcode.

FIG. 4 shows a heatmap plot for the assessment of DNA barcoding success in microparticles through split-and-pool ligations using Next Generation Sequencing (NGS). The sequencing read frequency for combinations of two DNA barcodes, bead barcode 1 (bb1) and bead barcode 2 (bb2), was calculated. The presence or absence of each combination is visualized in the heat map above. A total of 96 sequences for each barcode were analyzed, with combinations that are present indicated by grey squares, while missing combinations are shown in white. This plot illustrates successful ligations, as no missing combinations were observed in the sequenced library sample.

FIG. 5 shows pictures of microparticles loaded with C terminal GST tagged proteins after the proteins were incubated with microparticles harboring polyclonal anti GST antibodies on their surface. The presence and the level of recombinant protein loaded on the surface of the microparticles was assayed by a FITC labeled anti GST antibody. The figure shows images in FITC channel and brightfield.

FIG. 6 shows pictures of microparticle emulsion droplets and their stability post PCR amplification. Barcoded microparticle loaded with recombinant proteins were emulsified, subjected to 35 cycles of PCR and the stability of the emulsion was assayed by optical microscopy. The analysis of the post PCR emulsion droplet shows minimal failure due to droplets coalescence.

FIG. 7A-C shows a graphic of the steps of barcoding microparticles. FIG. 7A shows Step 1: a microparticle functionalized with two orthogonal click chemistry groups modified with a grafting primer A and with a group for the capture of a target molecule.

FIG. 7B shows a graphic of step 2 of barcoding microparticles. Microparticles are barcoded through sequential splint ligations of single stranded DNA oligonucleotide on a first functionalized group. PBC1: particle barcode 1, PBC2 particle barcode 2, TBC: target Barcode, B: Primer site B, A: grafting primer A.

FIG. 7C shows a graphic of step 3 of barcoding microparticles. Target molecules, for example proteins are immobilized on the second functionalized group.

FIG. 8A shows a graphic of a DEL selection assay with a bifunctionalized microparticle. In the first step target molecule (for example protein) coated microparticles are incubated with a DNA encoded library of molecules (DEL). Non specific binders are washed away, and microparticles are mixed with polymerase chain reaction reagents and compartmentalized by emulsion in fluorinated oil. PBC1: particle barcode 1, PBC2 particle barcode 2, TBC: target Barcode, B: Primer site B, A: grafting primer A, C primer site C, DEL BC: DNA Encoded Library barcode. FIG. 8B shows a graphic of the 2nd step of the DEL selection assay. In the step of Emulsion polymerase chain reaction the DNA encoded library members that bound to the target molecule on the microparticle are amplified with Primer B and C′. FIG. 8C shows a graphic of the 2nd step. After several amplification rounds, the PCR products of the DEL molecules are annealing onto the microparticle barcodes, allowing for a templated extension of the DEL barcode onto the microparticle barcode. FIG. 8D shows the particles loaded with the primer extended PCR product comprising the particle barcodes, the target barcode, and the DEL barcode in one molecule after successful emulsion PCR.

FIG. 8E shows a graphic of the last step of the DEL selection assay. Emulsion PCR droplets are collected, broken with perfluoro-octanol and the microparticles are collected for the downstream preparation of next generation sequencing libraries. A second PCR is used to add the sequencing adapters (SA1 and SA2).

FIG. 9 shows a graphic of a DEL selection assay with a bifunctionalized microparticle that includes an additional protein coding region (PC) encoding the target protein in the single stranded DNA oligonucleotide barcode DNA. The protein coding region allows direct synthesis of the target protein in an emulsion where it can bind directly to the microparticle.

FIG. 10 shows a schematic for the discovery of molecular glues. A series of three binding assays is performed and molecular glues are identified.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

The following definitions supplement those in the art and are directed to the current application and are not to be imputed to any related or unrelated cases, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a”, “an”, and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C”. The term “or” can be used conjunctively or disjunctively, unless the context specifically refers to a disjunctive use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)” this means a range whose lower limit is the first number and whose upper limit is the second number. For example, 25 to 100 mm means a range whose lower limit is 25 mm, and whose upper limit is 100 mm.

An “amplification sequence” as used herein, refers to a nucleic acid sequence that can be elongated or amplified by using a primer that hybridizes to said amplification sequence. An amplification sequence can be amplified, for example with a PCR reaction that uses a first amplification sequence and a second amplification sequence. Briefly, a first primer complementary to a first amplification sequence and a second primer complementary to a second amplification sequence can be used in a PCR reaction to amplify a sequence comprising at least the first amplification sequence and the second amplification sequence.

A “small molecule,” as used herein, refers to a molecule with a molecular weight less than 1000 daltons. In embodiments, the small molecule is an organic compound. In embodiments, the small molecule is capable of regulating a cell function or biological process.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. In embodiments, the peptide is at least 10 amino acids. In embodiments, the peptide is at least 20 amino acids. In embodiments, the peptide is at least 30 amino acids. In embodiments, the peptide is at least 40 amino acids. In embodiments, the peptide is at least 50 amino acids. In embodiments, the peptide is at least 60 amino acids. In embodiments, the peptide is at least 70 amino acids. In embodiments, the peptide is at least 80 amino acids. In embodiments, the peptide is at least 90 amino acids. In embodiments, the peptide is at least 100 amino acids. In embodiments, the peptide is about 10-1000 amino acids. In embodiments, the peptide is about 10-800 amino acids. In embodiments, the peptide is about 10-500 amino acids. In embodiments, the peptide is about 20-1000 amino acids. In embodiments, the peptide is about 20-800 amino acids. In embodiments, the peptide is about 20-500 amino acids.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that match (i.e., about 60% matching, preferably 65%, 70%, 75%, 80%, 85%, 90%, 91%, 92%, 93%, 94%, 95%, 96%, 97%, 98%, 99%, or higher over a specified region). In embodiments, a complement includes a nucleic acid sequence where at least 65% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 70% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 75% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 80% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 85% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 90% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 95% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 96% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 97% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 98% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where at least 99% of the nucleotides base pair with corresponding complementary nucleotides of a second nucleic acid sequence (e.g. where the complement is the complement of the second hybridization sequence and the second nucleic acid is the first hybridization sequence as disclosed herein). In embodiments, a complement includes a nucleic acid sequence where 100% of the nucleotides base pair with corresponding complementary nucleotides of a second nucleic acid sequence (e.g. where the complement is the complement of the second hybridization sequence and the second nucleic acid is the first hybridization sequence as disclosed herein).

By “binding” is meant attaching by a covalent bond or a non-covalent bond. Non-covalent bonds include those formed by van der Waals forces, hydrogen bonds, ionic bonds, entrapment or physical encapsulation, absorption, adsorption, and/or other intermolecular forces. Binding can be effectuated by any useful means, such as by enzymatic binding (e.g., enzymatic ligation) or by chemical binding (e.g., chemical ligation).

The term “aptamer” as provided herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLOS ONE 5 (12): e15004). Applying the SELEX and the SOMAmer technology includes for instance adding functional groups that mimic amino acid side chains to expand the aptamer's chemical diversity. As a result high affinity aptamers for almost any protein target are enriched and identified. Aptamers exhibit many desirable properties for targeted drug delivery, such as ease of selection and synthesis, high binding affinity and specificity, flexible structure, low immunogenicity, and versatile synthetic accessibility.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

An “inhibitor” refers to a compound (e.g., compounds described herein) that reduces activity when compared to a control, such as absence of the compound or a compound with known inactivity.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., a protein associated disease, a cancer (e.g., cancer, inflammatory disease, autoimmune disease, or infectious disease)) means that the disease (e.g., cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

In this disclosure, “comprises,” “comprising,” “containing” and “having” and the like can have the meaning ascribed to them in U.S. patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “nucleic acid multifunctional sequence” as used herein is a nucleic acid sequence with multiple functions. For example, the nucleic acid multifunctional sequence can contain an attachment point to a microparticle. The nucleic acid multifunctional sequence can contain a nucleic acid target molecule identifier sequence. The nucleic acid multifunctional sequence can contain a nucleic acid polypeptide identifier sequence. The nucleic acid multifunctional sequence can contain a polypeptide encoding nucleic acid sequence. In embodiments, the nucleic acid multifunctional sequence contains DNA, RNA, or a mixture of DNA and RNA. The nucleic acid multifunctional sequence can contain primer binding sites that can be used to amplify all or parts of the nucleic acid multifunctional sequence. In embodiments, the nucleic acid multifunctional sequence is a single strand nucleic acid.

A “nucleic acid target molecule identifier sequence” is a nucleic acid sequence that can be used to identify a target molecule. A target molecule, for example in a target molecule library, is assigned a unique nucleic acid target molecule identifier sequence. By de-coding the nucleic acid target molecule identifier sequence each associated target molecule can be unambiguously identified. For example, all or a part of a nucleic acid target molecule identifier sequence can be amplified and sequenced to identify the assigned target molecule.

A “target molecule” is a molecule that can be used to identify binders to the target molecule. In embodiments, the target molecule is a biomolecule. In embodiments, the target molecule is a peptide, polypeptide or protein. In embodiments, the target molecule is a peptide that forms part of a protein complex. As used herein, the term “biomolecule” refers to an agent (e.g., a compound, macromolecule, or small molecule), and the like derived from a biological system (e.g., an organism). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, a carbohydrate, a lipid, a protein, or any combination thereof. In some instances, the biomolecule may include one or more constituents of a cell but may not include other constituents of the cell. In embodiments, a biomolecule is a molecule produced by a biological system (e.g., an organism).

Non-limiting examples of biomolecules include, but are not limited to, polymers (e.g., natural or synthetic), lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In embodiments, a biomolecules is a peptide or protein (e.g., antibody and/or enzyme). In embodiments, a biomolecules is a nucleic acid.

Biomolecules may be labeled and referred to herein as a “labeled biomolecule”. A “labeled biomolecule” is bound either covalently through a linker or a chemical bond, or non-covalently, through van der Waals bonds, electrostatic bonds or hydrogen bonds to a label such that the presence of the labeled biomolecule (e.g., protein, polypeptide, nucleic acid, polymer) may be detected by detecting the presence of the label attached to the labeled biomolecule.

A “nucleic acid polypeptide identifier sequence” is a nucleic acid sequence that can be used to identify a target polypeptide. A target polypeptide, for example in a target polypeptide library, is assigned a unique nucleic acid polypeptide identifier sequence. By de-coding the nucleic acid polypeptide identifier sequence each associated target polypeptide can be unambiguously identified. For example, all or a part of a nucleic acid polypeptide identifier sequence can be amplified and sequenced to identify the assigned target polypeptide.

A “polypeptide encoding nucleic acid sequence” is a nucleic acid encoding a target polypeptide. For example, the target polypeptide encoding nucleic acid sequence can be transcribed and/or translated into a target polypeptide sequence.

A “nucleic acid compound identifier sequence” is a nucleic acid sequence that can be used to identify a compound. A compound, for example in a compound library, is assigned a unique nucleic acid compound identifier sequence. By de-coding the nucleic acid compound identifier sequence each associated compound can be unambiguously identified. For example, all or a part of a nucleic acid compound identifier sequence can be amplified and sequenced to identify the assigned compound.

A “DNA compound sequence” is a nucleic acid sequence with multiple functions. A DNA compound sequence contains a nucleic acid compound identifier sequence and a hybridization sequence. In embodiments, the DNA compound sequence contains DNA, RNA, or a mixture of DNA and RNA. The DNA compound sequence can contain primer binding sites that can be used to amplify all or parts of the DNA compound sequence.

A “hybridization sequence” is a nucleic acid sequence that is complementary to and can be hybridized with a second hybridization sequence. Hybridization can occur, for example, after melting and annealing of two single stranded nucleic acid strands (e.g., DNA). In some embodiments, the hybridization is along the full length of the hybridization sequences. In some embodiments, the hybridization is a partial hybridization between the hybridization sequences. In some embodiments, the hybridization is at the 5′ end, 3′ end, or in the middle of the hybridization sequence.

A “microparticle,” as used herein, is a particle wherein the longest diameter is less than or equal to 500 micrometer. The longest dimension of the microparticle may be referred to herein as the length of the microparticle. The shortest dimension of the microparticle may be referred to herein refer as the width of the microparticle. Microparticles may be composed of any appropriate material. For example, microparticle cores may include appropriate metals and metal oxides thereof (e.g., a metal microparticle core), carbon (e.g., an organic microparticle core) silicon and oxides thereof (e.g., a silicon microparticle core) or boron and oxides thereof (e.g., a boron microparticle core), a hydrogel (e.g., a PEG hydrogel), or mixtures thereof. In embodiments, the microparticle has the shape of a sphere, rod, cube, triangular, hexagonal, cylinder, spherocylinder, or ellipsoid. Microparticles can be superparamagnetic. Microparticles can be dissolvable. Microparticles can be monodispers. Microparticles can be fluorescent. Microparticles can be hydrophilic. A microparticle can be solid.

### II. Compositions

Provided herein are, inter alia, compositions comprising multifunctional microparticles and multifunctional microparticles presenting nucleic acid encoded target molecules. The multifunctional microparticles can be used for screening compounds that are capable of binding the target molecules.

In one aspect, the disclosure provides for a composition comprising a microparticle; a target molecule bound to said microparticle; and a nucleic acid multifunctional sequence bound to said microparticle, wherein said nucleic acid multifunctional sequence comprises: a nucleic acid target molecule identifier sequence; and a first hybridization sequence.

In one aspect, the disclosure provides for a composition comprising a microparticle; a target polypeptide bound to said microparticle; and a nucleic acid multifunctional sequence bound to said microparticle, wherein said nucleic acid multifunctional sequence comprises: a nucleic acid polypeptide identifier sequence; and a first hybridization sequence.

In one aspect, the disclosure provides for a composition comprising a microparticle; a target molecule bound to said microparticle; a nucleic acid multifunctional sequence bound to said microparticle, wherein said nucleic acid multifunctional sequence comprises: a nucleic acid target molecule identifier sequence; a first hybridization sequence; and a DNA encoded compound bound to said target molecule, said DNA encoded compound comprising a compound bound to a DNA compound sequence, said DNA compound sequence comprising: a nucleic acid compound identifier sequence; and a second hybridization sequence wherein a complement of the second hybridization sequence is capable of hybridizing to said first hybridization sequence.

In one aspect, the disclosure provides for a composition comprising a microparticle; a target molecule bound to said microparticle; a nucleic acid multifunctional sequence bound to said microparticle, wherein said nucleic acid multifunctional sequence comprises: a nucleic acid target molecule identifier sequence; a first hybridization sequence; and a DNA encoded compound bound to said nucleic acid multifunctional sequence, said DNA encoded compound comprising a compound bound to a DNA compound sequence, said DNA compound sequence comprising: a nucleic acid compound identifier sequence; and a complement of a second hybridization sequence, wherein the complement of the second hybridization sequence and the first hybridization sequence are bound to each other.

In some embodiments, the target molecule is a polypeptide.

In one aspect, the disclosure provides for a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; and a first hybridization sequence; and a DNA encoded compound bound to said target polypeptide, said DNA encoded compound comprising a compound bound to a DNA compound sequence, said DNA compound sequence comprising: a nucleic acid compound identifier sequence; and a second hybridization sequence wherein a complement of the second hybridization sequence is capable of hybridizing to said first hybridization sequence.

In one aspect, the disclosure provides for a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; a target polypeptide encoding nucleic acid sequence; and a first hybridization sequence; and a DNA encoded compound bound to said target polypeptide, said DNA encoded compound comprising a compound bound to a DNA compound sequence, said DNA compound sequence comprising: a nucleic acid compound identifier sequence; and a second hybridization sequence wherein a complement of the second hybridization sequence is capable of hybridizing to said first hybridization sequence.

In one aspect, the disclosure provides for a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; a first hybridization sequence; and a DNA encoded compound bound to said nucleic acid multifunctional sequence, said DNA encoded compound comprising a compound bound to a DNA compound sequence, said DNA compound sequence comprising: a nucleic acid compound identifier sequence; and a second hybridization sequence, wherein a complement of the second hybridization sequence and the first hybridization sequence are bound to each other.

In one aspect, the disclosure provides for a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; a target polypeptide encoding nucleic acid sequence; a first hybridization sequence; and a DNA encoded compound bound to said nucleic acid multifunctional sequence, said DNA encoded compound comprising a compound bound to a DNA compound sequence, said DNA compound sequence comprising: a nucleic acid compound identifier sequence; and a second hybridization sequence, wherein a complement of the second hybridization sequence and the first hybridization sequence are bound to each other.

In one aspect, the disclosure provides for a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; and a first hybridization sequence; and a DNA encoded compound, said DNA encoded compound comprising a compound bound to a DNA compound sequence, said DNA compound sequence comprising: a nucleic acid compound identifier sequence; and a second hybridization sequence wherein a complement of the second hybridization sequence is capable of hybridizing to said first hybridization sequence; wherein the microparticle and the DNA encoded compound are encapsulated in the same compartment.

In one aspect, the disclosure provides for a composition comprising a microparticle; a target polypeptide bound to said microparticle; a nucleic acid multifunctional sequence bound to said microparticle, wherein said nucleic acid multifunctional sequence comprises: a nucleic acid polypeptide identifier sequence; and a first hybridization sequence; and a DNA encoded compound, said DNA encoded compound comprising a compound bound to a DNA compound sequence, said DNA compound sequence comprising: a nucleic acid compound identifier sequence; and a second hybridization sequence wherein a complement of the second hybridization sequence is capable of hybridizing to said first hybridization sequence; wherein the microparticle and the DNA encoded compound are encapsulated in the same compartment.

In one aspect, the disclosure provides for a composition comprising a solid microparticle; a polypeptide bound to said solid microparticle; a nucleic acid multifunctional sequence bound to said microparticle, wherein said nucleic acid multifunctional sequence comprises: a nucleic acid polypeptide identifier sequence; a polypeptide encoding nucleic acid sequence; and a first hybridization sequence; and a DNA encoded compound bound to said polypeptide, said DNA encoded compound comprising a compound bound to a DNA compound sequence, said DNA compound sequence comprising: a nucleic acid compound identifier sequence; and a second hybridization sequence capable of hybridizing to said first hybridization sequence.

In one aspect, the disclosure provides for a composition comprising a solid microparticle; a polypeptide bound to said solid microparticle; a nucleic acid multifunctional sequence bound to said microparticle, wherein said nucleic acid multifunctional sequence comprises: a nucleic acid polypeptide identifier sequence; a polypeptide encoding nucleic acid sequence; a first hybridization sequence; and a DNA encoded compound bound to said nucleic acid multifunctional sequence, said DNA encoded compound comprising a compound bound to a DNA compound sequence, said DNA compound sequence comprising: a nucleic acid compound identifier sequence; and a second hybridization sequence, wherein the second hybridization sequence and the first hybridization sequence are bound to each other.

In one aspect, the disclosure provides for a composition comprising a solid microparticle; a polypeptide bound to said solid microparticle; a nucleic acid multifunctional sequence bound to said microparticle, wherein said nucleic acid multifunctional sequence comprises: a nucleic acid polypeptide identifier sequence; a polypeptide encoding nucleic acid sequence; and a first hybridization sequence; and a DNA encoded compound, said DNA encoded compound comprising a compound bound to a DNA compound sequence, said DNA compound sequence comprising: a nucleic acid compound identifier sequence; and a second hybridization sequence capable of hybridizing to said first hybridization sequence; wherein the microparticle and the DNA encoded compound are encapsulated in the same compartment.

In some embodiments, the nucleic acid multifunctional sequence further comprises a target polypeptide encoding nucleic acid sequence.

In some embodiments, the nucleic acid multifunctional sequence further comprises a bead identification sequence.

In some embodiments, the composition further comprises a nucleic acid identification sequence. In aspects the nucleic acid identification sequence comprises the nucleic acid target molecule identifier sequence or nucleic acid polypeptide identifier sequence, the first hybridization sequence, and the nucleic acid compound identifier sequence.

In some embodiments, the nucleic acid identification sequence is between 2 and 100 bases long. In some embodiments, the nucleic acid identification sequence is between 10 and 100 bases long. In some embodiments, the nucleic acid identification sequence is between 20 and 100 bases long. In some embodiments, the nucleic acid identification sequence is between 30 and 100 bases long. In some embodiments, the nucleic acid identification sequence is between 40 and 100 bases long. In some embodiments, the nucleic acid identification sequence is between 50 and 100 bases long. In some embodiments, the nucleic acid identification sequence is between 60 and 100 bases long. In some embodiments, the nucleic acid identification sequence is between 70 and 100 bases long. In some embodiments, the nucleic acid identification sequence is between 80 and 100 bases long. In some embodiments, the nucleic acid identification sequence is between 90 and 100 bases long. In some embodiments, the nucleic acid identification sequence is about 2 bases long. In some embodiments, the nucleic acid identification sequence is about 3 bases long. In some embodiments, the nucleic acid identification sequence is about 4 bases long. In some embodiments, the nucleic acid identification sequence is about 5 bases long. In some embodiments, the nucleic acid identification sequence is about 6 bases long. In some embodiments, the nucleic acid identification sequence is about 7 bases long. In some embodiments, the nucleic acid identification sequence is about 8 bases long. In some embodiments, the nucleic acid identification sequence is about 9 bases long. In some embodiments, the nucleic acid identification sequence is about 10 bases long. In some embodiments, the nucleic acid identification sequence is about 20 bases long. In some embodiments, the nucleic acid identification sequence is about 30 bases long. In some embodiments, the nucleic acid identification sequence is about 40 bases long. In some embodiments, the nucleic acid identification sequence is about 50 bases long. In some embodiments, the nucleic acid identification sequence is about 60 bases long. In some embodiments, the nucleic acid identification sequence is about 70 bases long. In some embodiments, the nucleic acid identification sequence is about 80 bases long. In some embodiments, the nucleic acid identification sequence is about 90 bases long. In some embodiments, the nucleic acid identification sequence is about 100 bases long.

In some embodiments, the first hybridization sequence and the second hybridization sequence are 100% complementary. In some embodiments, the first hybridization sequence and the second hybridization sequence are 99% complementary. In some embodiments, the first hybridization sequence and the second hybridization sequence are 98% complementary. In some embodiments, the first hybridization sequence and the second hybridization sequence are 97% complementary. In some embodiments, the first hybridization sequence and the second hybridization sequence are 96% complementary. In some embodiments, the first hybridization sequence and the second hybridization sequence are 95% complementary. In some embodiments, the first hybridization sequence and the second hybridization sequence are 94% complementary. In some embodiments, the first hybridization sequence and the second hybridization sequence are 93% complementary. In some embodiments, the first hybridization sequence and the second hybridization sequence are 92% complementary. In some embodiments, the first hybridization sequence and the second hybridization sequence are 91% complementary. In some embodiments, the first hybridization sequence and the second hybridization sequence are 90% complementary.

In some embodiments, the first hybridization sequence and the second hybridization sequence are 100% identical. In some embodiments, the first hybridization sequence and the second hybridization sequence are 99% identical. In some embodiments, the first hybridization sequence and the second hybridization sequence are 98% identical. In some embodiments, the first hybridization sequence and the second hybridization sequence are 97% identical. In some embodiments, the first hybridization sequence and the second hybridization sequence are 96% identical. In some embodiments, the first hybridization sequence and the second hybridization sequence are 95% identical. In some embodiments, the first hybridization sequence and the second hybridization sequence are 94% identical. In some embodiments, the first hybridization sequence and the second hybridization sequence are 93% c identical. In some embodiments, the first hybridization sequence and the second hybridization sequence are 92% identical. In some embodiments, the first hybridization sequence and the second hybridization sequence are 91% identical. In some embodiments, the first hybridization sequence and the second hybridization sequence are 90% identical.

In some embodiments, the first hybridization sequence and the complement of the second hybridization sequence are 100% complementary. In some embodiments, the first hybridization sequence and the complement of the second hybridization sequence are 99% complementary. In some embodiments, the first hybridization sequence and the complement of the second hybridization sequence are 98% complementary. In some embodiments, the first hybridization sequence and the complement of the second hybridization sequence are 97% complementary. In some embodiments, the first hybridization sequence and the complement of the second hybridization sequence are 96% complementary. In some embodiments, the first hybridization sequence and the complement of the second hybridization sequence are 95% complementary. In some embodiments, the first hybridization sequence and the complement of the second hybridization sequence are 94% complementary. In some embodiments, the first hybridization sequence and the complement of the second hybridization sequence are 93% complementary. In some embodiments, the first hybridization sequence and the complement of the second hybridization sequence are 92% complementary. In some embodiments, the first hybridization sequence and the complement of the second hybridization sequence are 91% complementary. In some embodiments, the first hybridization sequence and the complement of the second hybridization sequence are 90% complementary.

In some embodiments, the first hybridization sequence and the second hybridization sequence have a melting temperature when annealed of about 60° C. In some embodiments, the first hybridization sequence and the second hybridization sequence have a melting temperature when annealed of about 61° C. In some embodiments, the first hybridization sequence and the second hybridization sequence have a melting temperature when annealed of about 62° C. In some embodiments, the first hybridization sequence and the second hybridization sequence have a melting temperature when annealed of about 63° C. In some embodiments, the first hybridization sequence and the second hybridization sequence have a melting temperature when annealed of about 64° C. In some embodiments, the first hybridization sequence and the second hybridization sequence have a melting temperature when annealed of about 65° C. In some embodiments, the first hybridization sequence and the second hybridization sequence have a melting temperature when annealed of about 66° C. In some embodiments, the first hybridization sequence and the second hybridization sequence have a melting temperature when annealed of about 67° C. In some embodiments, the first hybridization sequence and the second hybridization sequence have a melting temperature when annealed of about 68° C. In some embodiments, the first hybridization sequence and the second hybridization sequence have a melting temperature when annealed of about 69° C. In some embodiments, the first hybridization sequence and the second hybridization sequence have a melting temperature when annealed of about 70° C. In some embodiments, the first hybridization sequence and the second hybridization sequence have a melting temperature when annealed of about 71° C. In some embodiments, the first hybridization sequence and the second hybridization sequence have a melting temperature when annealed of about 72° C. In some embodiments, the first hybridization sequence and the second hybridization sequence have a melting temperature when annealed of about 73° C. In some embodiments, the first hybridization sequence and the second hybridization sequence have a melting temperature when annealed of about 74° C. In some embodiments, the first hybridization sequence and the second hybridization sequence have a melting temperature when annealed of about 75° C.

In some embodiments, the first hybridization sequence and the complement of the second hybridization sequence have a melting temperature when annealed of about 60° C. In some embodiments, the first hybridization sequence and the complement of the second hybridization sequence have a melting temperature when annealed of about 61° C. In some embodiments, the first hybridization sequence and the complement of the second hybridization sequence have a melting temperature when annealed of about 62° C. In some embodiments, the first hybridization sequence and the complement of the second hybridization sequence have a melting temperature when annealed of about 63° C. In some embodiments, the first hybridization sequence and the complement of the second hybridization sequence have a melting temperature when annealed of about 64° C. In some embodiments, the first hybridization sequence and the complement of the second hybridization sequence have a melting temperature when annealed of about 65° C. In some embodiments, the first hybridization sequence and the complement of the second hybridization sequence have a melting temperature when annealed of about 66° C. In some embodiments, the first hybridization sequence and the complement of the second hybridization sequence have a melting temperature when annealed of about 67° C. In some embodiments, the first hybridization sequence and the complement of the second hybridization sequence have a melting temperature when annealed of about 68° C. In some embodiments, the first hybridization sequence and the complement of the second hybridization sequence have a melting temperature when annealed of about 69° C. In some embodiments, the first hybridization sequence and the complement of the second hybridization sequence have a melting temperature when annealed of about 70° C. In some embodiments, the first hybridization sequence and the complement of the second hybridization sequence have a melting temperature when annealed of about 71° C. In some embodiments, the first hybridization sequence and the complement of the second hybridization sequence have a melting temperature when annealed of about 72° C. In some embodiments, the first hybridization sequence and the complement of the second hybridization sequence have a melting temperature when annealed of about 73° C. In some embodiments, the first hybridization sequence and the complement of the second hybridization sequence have a melting temperature when annealed of about 74° C. In some embodiments, the first hybridization sequence and the complement of the second hybridization sequence have a melting temperature when annealed of about 75° C.

In some embodiments, the nucleic acid multifunctional sequence is bound to the microparticle by a nucleic acid linker. In some embodiments, the nucleic acid linker is about 1-100 bases long. In some embodiments, the nucleic acid linker is about 1 base long. In some embodiments, the nucleic acid linker is about 2 bases long. In some embodiments, the nucleic acid linker is about 3 bases long. In some embodiments, the nucleic acid linker is about 4 bases long. In some embodiments, the nucleic acid linker is about 5 bases long. In some embodiments, the nucleic acid linker is about 6 bases long. In some embodiments, the nucleic acid linker is about 7 bases long. In some embodiments, the nucleic acid linker is about 8 bases long. In some embodiments, the nucleic acid linker is about 9 bases long. In some embodiments, the nucleic acid linker is about 10 bases long. In some embodiments, the nucleic acid linker is about 20 bases long. In some embodiments, the nucleic acid linker is about 30 bases long. In some embodiments, the nucleic acid linker is about 40 bases long. In some embodiments, the nucleic acid linker is about 50 bases long. In some embodiments, the nucleic acid linker is about 60 bases long. In some embodiments, the nucleic acid linker is about 70 bases long. In some embodiments, the nucleic acid linker is about 80 bases long. In some embodiments, the nucleic acid linker is about 90 bases long. In some embodiments, the nucleic acid linker is about 100 bases long.

In some embodiments, the nucleic acid multifunctional sequence is bound to the microparticle by a chemical linker.

In some embodiments, the nucleic acid multifunctional sequence further comprises one or more unique molecular identifier (UMI). In some embodiments, the UMI encodes for the target polypeptide. In some embodiments, the UMI encodes for the bead. In some embodiments, the UMI encodes for the experiment.

In some embodiments, the nucleic acid multifunctional sequence is a DNA, RNA, or a mixture of DNA and RNA. In some embodiments, the nucleic acid multifunctional sequence is a DNA. In some embodiments, the nucleic acid multifunctional sequence is a RNA. In some embodiments, the nucleic acid multifunctional sequence is a mixture of DNA and RNA. In some embodiments, the nucleic acid multifunctional sequence is a PNA. In some embodiments, the nucleic acid multifunctional sequence is alternate genetic polymer, for example as described in Pinheiro et al., Science (2012) 336 (6079): 341-344.

In some embodiments, the nucleic acid multifunctional sequence is a single stranded nucleic acid. In some embodiments, the nucleic acid multifunctional sequence is a double stranded nucleic acid. In some embodiments, the nucleic acid multifunctional sequence is a double stranded nucleic acid and the 5′ and 3′ ends each comprise a hairpin of unpaired bases and the hairpin connects the 5′ end of one strand to the 3′ end of the other strand.

In some embodiments, the hairpins are at least 3 bases long. In some embodiments, the hairpins are at least 4 bases long. In some embodiments, the hairpins are at least 5 bases long. In some embodiments, the hairpins are at least 6 bases long. In some embodiments, the hairpins are at least 7 bases long. In some embodiments, the hairpins are at least 8 bases long. In some embodiments, the hairpins are at least 9 bases long. In some embodiments, the hairpins are at least 10 bases long. In some embodiments, the hairpins are at least 11 bases long. In some embodiments, the hairpins are at least 12 bases long. In some embodiments, the hairpins are at least 13 bases long. In some embodiments, the hairpins are at least 14 bases long. In some embodiments, the hairpins are at least 15 bases long. In some embodiments, the hairpins are at least 16 bases long. In some embodiments, the hairpins are at least 17 bases long. In some embodiments, the hairpins are at least 18 bases long. In some embodiments, the hairpins are at least 19 bases long. In some embodiments, the hairpins are at least 20 bases long. In some embodiments, the hairpins are at least 30 bases long. In some embodiments, the hairpins are at least 40 bases long. In some embodiments, the hairpins are at least 50 bases long. In some embodiments, the hairpins are between 3 and 50 bases long. In some embodiments, the hairpins are between 3 and 40 bases long. In some embodiments, the hairpins are between 3 and 30 bases long. In some embodiments, the hairpins are between 3 and 20 bases long. In some embodiments, the hairpins are between 3 and 10 bases long. In some embodiments, the hairpins are between 10 and 50 bases long. In some embodiments, the hairpins are between 10 and 40 bases long. In some embodiments, the hairpins are between 10 and 30 bases long. In some embodiments, the hairpins are between 10 and 20 bases long. In some embodiments, the hairpins are the same length. In some embodiments, the hairpins are different length. In some embodiments, the hairpins have the same sequence. In some embodiments, the hairpins have a different sequence.

In some embodiments, the nucleic acid multifunctional sequence further comprises a first amplification sequence.

In some embodiments, the DNA compound sequence further comprises a second amplification sequence.

In some embodiments, the nucleic acid multifunctional sequence and/or the DNA compound sequence is resistant to nuclease degradation. In some embodiments, the nucleic acid multifunctional sequence and/or the DNA compound sequence comprises modified bases or linkages.

In some embodiments, the nucleic acid multifunctional sequence and/or the DNA compound sequence comprises one or more chemically modified nucleoside. A chemically modified nucleoside may comprise a modified sugar moiety and/or a modified nucleobase. In some embodiments, a chemically modified nucleosides disclosed may be a locked nucleic acid (LNA), e.g., LNA cytidine, LNA thymidine, LNA adenosine, or LNA guanosine. In some embodiments, chemically modified nucleosides comprise 2′-O-methyl (“2′-OMe”) ribonucleosides, for example, 2′-O-methylcytidine, 2′-O-methylguanosine, 2′-O-methylthymidine, 2′-O-methyluridine, and/or 2′-O-methyladenosine. In some embodiments, a chemically modified nucleoside comprises a 5-methylpyrimidine, for example, 5-methylcytosine; and/or a 5-methylpurine, for example, 5-methylguanine. In some embodiments, chemically modified nucleosides can include any of the following chemically-modified nucleosides: 5-methyl-2′-O-methylcytidine, 5-methyl-2′-O-methylthymidine, 5-methylcytidine, 5-methyluridine, and/or 5-methyl-2′-deoxycytidine. In some embodiments, chemically modified nucleosides can include a stabilized terminal 5′-phosphate or phosphatase-resistant analogue of 5′-phosphate, e.g., 5′-methyl phosphonate, 5′-methylenephosphonate, a 5′-methylenephosphonate analog, 5′-E-vinyl phosphonate (5′-E-VP), 5′-phosphorothioate, and a 5′-C-methyl analog; a 2′-O-methyl ribonucleoside, e.g., 2′-O-methylcytidine, 2′-O-methylguanosine, 2′-O-methyluridine, 2′-O-methylthymidine, and 2′-O-methyladenosine; a 5-methylpyrimidine, e.g., 5-methylcytosine; a 5-methylpurine, e.g., 5-methylguanine; 5-methyl-2′-O-methylcytidine; 5-methyl-2′-O-methylthymidine; 5-methylcytidine; 5-methyluridine; or 5-methyl 2′-deoxycytidine.

In some embodiments, chemically modified nucleosides described herein include 2′-O-(2-methoxyethyl) (“2′-MOE”) nucleosides, 2′-deoxy-2′-fluoro nucleosides, 2′-fluoro-β-D-arabinonucleosides, bridged nucleosides, LNA nucleosides, constrained ethyl (CET) nucleosides, tricyclo-DNA (tcDNA) nucleosides, 2′-0,4′-C-ethylene linked nucleic acid (ENA) nucleosides, and/or peptide nucleic acids (PNA).

In some embodiments, the nucleic acid multifunctional sequence and/or the DNA compound sequence comprises a modified internucleoside linkage. In some embodiments, a modified internucleoside linkage includes, but is not limited to: a phosphorothioate linkage; a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, or a boranophosphate linkage.

In some embodiments, the nucleic acid multifunctional sequence and/or the DNA compound sequence comprises a ribose modification capable of increasing stability and/or activity relative to an unmodified nucleic acid. In some embodiments, the nucleic acid multifunctional sequence and/or the DNA compound sequence comprises a 2′-OMe or 2′-F modified ribose group. In some embodiments, the nucleic acid multifunctional sequence and/or the DNA compound sequence comprises one or more phosphorothioate linkages between nucleotides.

In some embodiments, the target polypeptide encoding nucleic acid sequence encodes all or a part of the target polypeptide. In some embodiments, the target polypeptide encoding nucleic acid sequence is about 90 to about 5000 bases long. In some embodiments, the target polypeptide encoding nucleic acid sequence is about 90 to about 4000 bases long. In some embodiments, the target polypeptide encoding nucleic acid sequence is about 90 to about 3000 bases long. In some embodiments, the target polypeptide encoding nucleic acid sequence is about 90 to about 2000 bases long. In some embodiments, the target polypeptide encoding nucleic acid sequence is about 90 to about 1000 bases long. In some embodiments, the target polypeptide encoding nucleic acid sequence is about 90 bases long. In some embodiments, the target polypeptide encoding nucleic acid sequence is about 90 bases long. In some embodiments, the target polypeptide encoding nucleic acid sequence is about 100 bases long.

In some embodiments, the target polypeptide encoding nucleic acid sequence is about 150 bases long. In some embodiments, the target polypeptide encoding nucleic acid sequence is about 200 bases long. In some embodiments, the target polypeptide encoding nucleic acid sequence is about 250 bases long. In some embodiments, the target polypeptide encoding nucleic acid sequence is about 300 bases long. In some embodiments, the target polypeptide encoding nucleic acid sequence is about 350 bases long. In some embodiments, the target polypeptide encoding nucleic acid sequence is about 400 bases long. In some embodiments, the target polypeptide encoding nucleic acid sequence is about 450 bases long. In some embodiments, the target polypeptide encoding nucleic acid sequence is about 500 bases long. In some embodiments, the target polypeptide encoding nucleic acid sequence is about 550 bases long. In some embodiments, the target polypeptide encoding nucleic acid sequence is about 600 bases long. In some embodiments, the target polypeptide encoding nucleic acid sequence is about 650 bases long. In some embodiments, the target polypeptide encoding nucleic acid sequence is about 700 bases long. In some embodiments, the target polypeptide encoding nucleic acid sequence is about 750 bases long. In some embodiments, the target polypeptide encoding nucleic acid sequence is about 800 bases long. In some embodiments, the target polypeptide encoding nucleic acid sequence is about 850 bases long. In some embodiments, the target polypeptide encoding nucleic acid sequence is about 900 bases long. In some embodiments, the target polypeptide encoding nucleic acid sequence is about 950 bases long. In some embodiments, the target polypeptide encoding nucleic acid sequence is about 1000 bases long. In some embodiments, the target polypeptide encoding nucleic acid sequence is about 2000 bases long. In some embodiments, the target polypeptide encoding nucleic acid sequence is about 3000 bases long. In some embodiments, the target polypeptide encoding nucleic acid sequence is about 4000 bases long. In some embodiments, the target polypeptide encoding nucleic acid sequence is about 5000 bases long.

In some embodiments, the longest dimension of the microparticle is at least 1 μm. In some embodiments, the microparticle is at least 2 μm. In some embodiments, the longest dimension of the microparticle is at least 3 μm. In some embodiments, the longest dimension of the microparticle is at least 4 μm. In some embodiments, the longest dimension of the microparticle is at least 5 μm. In some embodiments, the longest dimension of the microparticle is at least 6 μm. In some embodiments, the longest dimension of the microparticle is at least 7 μm. In some embodiments, the longest dimension of the microparticle is at least 8 μm. In some embodiments, the longest dimension of the microparticle is at least 9 μm. In some embodiments, the longest dimension of the microparticle is at least 10 μm. In some embodiments, the longest dimension of the microparticle is at least 15 μm. In some embodiments, the longest dimension of the microparticle is at least 20 μm. In some embodiments, the longest dimension of the microparticle is at least 30 μm. In some embodiments, the longest dimension of the microparticle is at least 40 μm. In some embodiments, the longest dimension of the microparticle is at least 50 μm. In some embodiments, the longest dimension of the microparticle is at least 60 μm. In some embodiments, the longest dimension of the microparticle is at least 70 μm. In some embodiments, the longest dimension of the microparticle is at least 50 μm. In some embodiments, the longest dimension of the microparticle is at least 60 μm. In some embodiments, the longest dimension of the microparticle is at least 70 μm. In some embodiments, the longest dimension of the microparticle is at least 80 μm. In some embodiments, the longest dimension of the microparticle is at least 90 μm. In some embodiments, the longest dimension of the microparticle is at least 100 μm. In some embodiments, the longest dimension of the microparticle is at least 150 μm. In some embodiments, the longest dimension of the microparticle is at least 200 μm. In some embodiments, the longest dimension of the microparticle is at least 250 μm. In some embodiments, the longest dimension of the microparticle is at least 300 μm. In some embodiments, the longest dimension of the microparticle is at least 350 μm. In some embodiments, the longest dimension of the microparticle is at least 400 μm. In some embodiments, the longest dimension of the microparticle is at least 450 μm. In some embodiments, the longest dimension of the microparticle is at least 500 μm.

In some embodiments, the longest dimension of the microparticle is between 1 μm and 10 μm. In some embodiments, the longest dimension of the microparticle is between 2 μm and 9 μm. In some embodiments, the longest dimension of the microparticle is between 3 μm and 8 μm. In some embodiments, the longest dimension of the microparticle is between 6 μm and 7 μm. In some embodiments, the longest dimension of the microparticle is between 10 μm and 20 μm. In some embodiments, the longest dimension of the microparticle is between 20 μm and 30 μm. In some embodiments, the longest dimension of the microparticle is between 30 μm and 40 μm. In some embodiments, the longest dimension of the microparticle is between 40 μm and 50 μm. In some embodiments, the longest dimension of the microparticle is between 10 μm and 50 μm. In some embodiments, the longest dimension of the microparticle is between 10 μm and 100 μm. In some embodiments, the longest dimension of the microparticle is between 50 μm and 100 μm. In some embodiments, the longest dimension of the microparticle is between 70 μm and 100 μm. In some embodiments, the longest dimension of the microparticle is between 80 μm and 100 μm. In some embodiments, the longest dimension of the microparticle is between 90 μm and 100 μm. In some embodiments, the longest dimension of the microparticle is between 50 μm and 200 μm. In some embodiments, the longest dimension of the microparticle is between 50 μm and 300 μm. In some embodiments, the longest dimension of the microparticle is between 50 μm and 400 μm. In some embodiments, the longest dimension of the microparticle is between 50 μm and 400 μm. In some embodiments, the longest dimension of the microparticle is between 100 μm and 200 μm. In some embodiments, the longest dimension of the microparticle is between 100 μm and 300 μm. In some embodiments, the longest dimension of the microparticle is between 100 μm and 400 μm. In some embodiments, the longest dimension of the microparticle is between 100 μm and 500 μm. In some embodiments, the longest dimension of the microparticle is at most 500 μm.

In some embodiments, the microparticle is a bead. In some embodiments, the microparticle is a hydrogel. In some embodiments, the microparticle is a microsphere. In some embodiments, the microparticle contains a polymer. In some embodiments, the microparticle contains a glass. In some embodiments, the microparticle contains a metal. In some embodiments, the microparticle contains a ceramic. In some embodiments, the microparticle contains a synthetic polymer. In some embodiments, the microparticle contains a gel. In some embodiments, the microparticle contains a polystyrene core.

In some embodiments, the microparticle is a PEG particle. In some embodiments, the microparticle is a polyacrylamide (PAA) particle. In some embodiments, the microparticle is magnetic.

In some embodiments, the microparticle is functionalized. In some embodiments, the functionalization facilitates covalent bonds. In some embodiments, the functionalization facilitates non-covalent bonds. In some embodiments, microparticle is functionalized with a polypeptide binding moiety. In some embodiments, microparticle is functionalized with a polynucleotide binding moiety. In some embodiments, microparticle is functionalized with a polynucleotide binding moiety and a polypeptide binding moiety. In some embodiments, microparticle is functionalized with between 10⁵ to 10⁶ polynucleotide binding moieties. In some embodiments, microparticle is functionalized with between 10⁶ to 107 polypeptide binding moieties.

In some embodiments, multiple nucleic acid multifunctional sequences are attached to the microparticle. In some embodiments, about 10⁵ to 10⁶ of said nucleic acid multifunctional sequences are attached to the microparticle. In embodiments, the nucleic acid multifunctional sequences attached to the same microparticle are the same.

In some embodiments, multiple target molecules are attached to the microparticle. In some embodiments, about 10⁶ to 10⁹ of target molecules are attached to the microparticle. In embodiments, the target molecules attached to the same microparticle are the same.

In some embodiments, multiple target polypeptides are attached to the microparticle. In some embodiments, about 10⁶ to 10⁹ of target polypeptides are attached to the microparticle. In embodiments, the polypeptides attached to the same microparticle are the same.

In some embodiments, the polypeptide further comprises a protein tag. In some embodiments, the protein tag is a Polyhistidine (His-tag), Glutathione S-transferase (GST), FLAG tag, SPY tag, calmodulin binding domain (CBD), chitin binding domain (CBD), choline-binding domain (CBD), albumin-binding protein (ABP), Bacteriophage T7 epitope (T7-tag), bacteriophage V5 epitope (V5-tag), biotin-carboxy carrier protein (BCCP), bluetongue virus tag (B-tag), chloramphenicol acetyl transferase (CAT), cellulose binding domain (CBP), E2 epitope, galactose-binding protein (GBP), green fluorescent protein (GFP), Glu-Glu (EE-tag), human influenza hemagglutinin (HA), HaloTag, maltose-binding protein (MBP), streptavadin-binding peptide (SBP), Myc epitope tag, strep-tag, or biotin.

In some embodiments, the polypeptide is bound non-covalently to the microparticle. In some embodiments, the polypeptide is bound covalently to the microparticle. In some embodiments, the polypeptide is bound to the microparticle by a polypeptide binding moiety. In some embodiments, the polypeptide binding moiety is a divalent metal, glutathione, anti-FLAG antibody or fragment thereof, calmodulin, chitin, choline, anti-E2 antibody or a fragment thereof, cellulose, albumin, streptavidin, anti-bluetongue virus antibody or a fragment thereof, galactose, anti-GFP antibody or a fragment thereof, biotin, or maltose.

In embodiments, where a molecule (e.g., a biomolecule such as a polypeptide, polypeptide binding moiety, or nucleic acid) is covalently bound to a microparticle as described herein, the covalent attachment is achieved using bioconjugate chemistry as described herein. For example, the molecule may be covalently bound to the microparticle through a bioconjugate linker as described herein. In embodiments, the covalent linkage on the microparticle is formed through an DBCO, hydroxyl, or an amine. In embodiments, the covalent linkage on the microparticle is formed through an azide, a 5′ phosphate, a 3′ phosphate, an NH ester, or an amine. In embodiments, the covalent linkage is formed through click chemistry. In embodiments, two different covalent linkages are formed through two orthogonal click chemistries.

In some embodiments, the polypeptide binding moiety further comprises a linker. In some embodiments, the linker is a polypeptide linker or a nucleic acid linker. In some embodiments, the linker is about 1-100 amino acids long. In some embodiments, the linker is about 1 amino acid long. In some embodiments, the linker is about 2 amino acids long. In some embodiments, the linker is about 3 amino acids long. In some embodiments, the linker is about 4 amino acids long. In some embodiments, the linker is about 5 amino acids long. In some embodiments, the linker is about 6 amino acids long. In some embodiments, the linker is about 7 amino acids long. In some embodiments, the linker is about 8 amino acids long. In some embodiments, the linker is about 9 amino acids long. In some embodiments, the linker is about 10 amino acids long. In some embodiments, the linker is about 20 amino acids long. In some embodiments, the linker is about 30 amino acids long. In some embodiments, the linker is about 40 amino acids long. In some embodiments, the linker is about 50 amino acids long. In some embodiments, the linker is about 60 amino acids long. In some embodiments, the linker is about 70 amino acids long. In some embodiments, the linker is about 80 amino acids long. In some embodiments, the linker is about 90 amino acids long. In some embodiments, the linker is about 100 amino acids long.

In some embodiments, the linker is about 1-100 bases long. In some embodiments, the linker is about 1 base long. In some embodiments, the linker is about 2 bases long. In some embodiments, the linker is about 3 bases long. In some embodiments, the linker is about 4 bases long. In some embodiments, the linker is about 5 bases long. In some embodiments, the linker is about 6 bases long. In some embodiments, the linker is about 7 bases long. In some embodiments, the linker is about 8 bases long. In some embodiments, the linker is about 9 bases long. In some embodiments, the linker is about 10 bases long. In some embodiments, the linker is about 20 bases long. In some embodiments, the linker is about 30 bases long. In some embodiments, the linker is about 40 bases long. In some embodiments, the linker is about 50 bases long. In some embodiments, the linker is about 60 bases long. In some embodiments, the linker is about 70 bases long. In some embodiments, the linker is about 80 bases long. In some embodiments, the linker is about 90 bases long. In some embodiments, the linker is about 100 bases long.

In some embodiments, wherein the compound is selected from the group of a small molecule, a macromolecule, a peptide, a polypeptide, a protein, a protein complex, a nucleic acid, a lipid, a carbohydrate, a glycan, and a cell. In embodiments, the compound is a biomolecule.

In some embodiments, the compound is a small molecule (e.g., a kinase inhibitor). In some embodiments, the compound is a macromolecule (e.g., a fatty acid, a carbohydrate). In some embodiments, the compound is a peptide. In some embodiments, the peptide is a neoantigen peptide. In some embodiments, the compound is a protein. In some embodiments, the protein is a protein complex. In some embodiments, the compound is a nucleic acid. In some embodiments, the nucleic acid is a RNA, a DNA, or a mixture of RNA or DNA. In some embodiments, the nucleic acid is a RNA. In some embodiments, the nucleic acid is a DNA. In some embodiments, the nucleic acid is a mixture of RNA or DNA. In some embodiments, the nucleic acid is an aptamer. In some embodiments, the compound is a lipid. In some embodiments, the compound is a cell. In embodiments, the nucleic acid is a peptide nucleic acid (PNA) or other genetic polymer. In some embodiments, the compound is a carbohydrate

In some embodiments, the composition is encapsulated. In some embodiments, the composition is partitioned into a compartment. In some embodiments, the compartment is an aqueous droplet within an emulsion.

In some embodiments, the composition is partitioned into a droplet. In some embodiments, the compartment is an emulsion. In some embodiments, the emulsion is an oil in water emulsion. In some embodiments, the size of the compartment is between 0.1 μm. and 0.1 mm. In some embodiments, the size of the compartment is 100 μm. In some embodiments, the size of the compartment is 150 μm. In some embodiments, the size of the compartment is 200 μm. In some embodiments, the size of the compartment is less than 200 μm.

In some embodiments, the encapsulation comprises exactly one composition per compartment. In some embodiments, the encapsulation comprises a plurality of compositions in a plurality of compartments. In some embodiments, each composition of the plurality of compositions is partitioned in a compartment of the plurality of compartments. In some embodiments, the compartments are similar in size. In some embodiments, the compartments have a narrow size distribution.

In aspects, a library of the composition is provided comprising a plurality of compositions described herein.

In some embodiments, the library comprises a plurality of microparticles, wherein each microparticle has a plurality of the same target polypeptide. In some embodiments, the library comprises a plurality of microparticles, wherein each microparticle comprises a different target polypeptide. In some embodiments, the library comprises a plurality of microparticles, wherein each microparticle has a plurality of the same target polypeptide, and wherein each microparticle comprises a different target polypeptide.

In some embodiments, the library comprises a plurality of target molecules. In some embodiments, the polypeptides are the same target molecules. In some embodiments, the target molecules are different target molecules.

In some embodiments, the library comprises a plurality of polypeptides. In some embodiments, the polypeptides are the same polypeptide. In some embodiments, the polypeptides are different polypeptides.

In some embodiments, the library comprises a plurality of DNA encoded compounds. In some embodiments, the DNA encoded compounds are the same compound. In some embodiments, the DNA encoded compounds are different compounds.

In some embodiments, the library is in an emulsion. In some embodiments, each member of the library is encapsulated. In some embodiments, each member of the library is partitioned into a compartment. In some embodiments, each compartment comprises not more than a single microparticle. In some embodiments, the compartment is an emulsion. In some embodiments, the emulsion is an oil in water emulsion. In some embodiments, the library is in a vessel. In some embodiments, each member of the library is in a vessel. A vessel, for example can be a tube, a well in a plate, a well in a high-throughput plate, or a droplet.

In some embodiments, the polypeptide is derived from a transcriptome. In some embodiments, the plurality of polypeptides in the library is a transcriptome of a cell. In some embodiments, the transcriptome is from a human cell. In some embodiments, the transcriptome is from a diseased human cell. In some embodiments, the transcriptome is from a healthy human cell.

In some embodiments, the library comprises a DNA encoded library displaying a target molecule described herein and a plurality of compounds described herein.

In some embodiments, the library comprises a DNA encoded library displaying a plurality of target molecules described herein and a compound described herein.

In some embodiments, the library comprises a DNA encoded library displaying a plurality of target molecules described herein and a plurality of compounds described herein.

In some embodiments, the library comprises a DNA encoded library displaying a polypeptide described herein and a plurality of compounds described herein.

In some embodiments, the library comprises a DNA encoded library displaying a plurality of polypeptides described herein and a compound described herein.

In some embodiments, the library comprises a DNA encoded library displaying a plurality of polypeptides described herein and a plurality of compounds described herein.

In some embodiments, the plurality of compounds is about 2, about 10, about 100, about 103, about 104, about 10⁵, about 10⁶, or about 107, or about 108 compounds, or about 10⁹ compounds, or about 1010 compounds, or about 1011 compounds. In some embodiments, the plurality of compounds is about 2. In some embodiments, the plurality of compounds is about 10. In some embodiments, the plurality of compounds is about 100. In some embodiments, the plurality of compounds is about 103. In some embodiments, the plurality of compounds is about 104. In some embodiments, the plurality of compounds is about 10⁵. In some embodiments, the plurality of compounds is about 10⁶. In some embodiments, the plurality of compounds is about 107. In some embodiments, the plurality of compounds is about 108. In some embodiments, the plurality of compounds is about 10⁹. In some embodiments, the plurality of compounds is about 1010. In some embodiments, the plurality of compounds is about 1011. In some embodiments, the plurality of polypeptides is about 2, 10, 100, about 103, about 104, about 10⁵, about 10⁶, or about 107, about 108 polypeptides, or about 10⁹ polypeptides. In some embodiments, the plurality of polypeptides is about 2. In some embodiments, the plurality of polypeptides is about 10, In some embodiments, the plurality of polypeptides is about 100. In some embodiments, the plurality of polypeptides is about 103. In some embodiments, the plurality of polypeptides is about 104. In some embodiments, the plurality of polypeptides is about 10⁵. In some embodiments, the plurality of polypeptides is about 10⁶. In some embodiments, the plurality of polypeptides is about 107. In some embodiments, the plurality of polypeptides is about 108.

### III. Methods of Producing Compositions

Provided herein are, inter alia, methods of making the compositions and libraries of compositions described herein.

In aspects, a method of making a composition of the disclosure is provided, the method comprising combining a microparticle, a polypeptide binding moiety, the polypeptide, the nucleic acid multifunctional sequence and the DNA encoded compound under conditions sufficient for (i) attaching the polypeptide binding moiety and the nucleic acid multifunctional sequence to the microparticle, (ii) binding the polypeptide to the polypeptide binding moiety; and (iii) binding the DNA encoded compound to the polypeptide.

In aspects, a method of making a composition of the disclosure is provided, the method comprising combining a microparticle, a polypeptide binding moiety, the polypeptide, the nucleic acid multifunctional sequence and the DNA encoded compound under conditions sufficient for (i) attaching the polypeptide binding moiety and the nucleic acid multifunctional sequence to the microparticle, and (ii) binding the polypeptide to the polypeptide binding moiety.

In aspects, a method of making a composition of the disclosure is provided, the method comprising combining a microparticle, a target molecule binding moiety, the polypeptide, the nucleic acid multifunctional sequence and the DNA encoded compound under conditions sufficient for (i) attaching the target molecule binding moiety and the nucleic acid multifunctional sequence to the microparticle, (ii) binding the target molecule to the target molecule binding moiety; and (iii) binding the DNA encoded compound to the target molecule.

In aspects, a method of making a composition of the disclosure is provided, the method comprising combining a microparticle, a polypeptide binding moiety, the polypeptide, the nucleic acid multifunctional sequence and the DNA encoded compound under conditions sufficient for (i) attaching the polypeptide binding moiety and the nucleic acid multifunctional sequence to the microparticle, and (ii) binding the polypeptide to the polypeptide binding moiety.

In aspects, a method of making a composition of the disclosure is provided, the method comprising combining a microparticle, a polypeptide binding moiety, the polypeptide, the nucleic acid multifunctional sequence and the DNA encoded compound under conditions sufficient for (i) attaching the polypeptide binding moiety and the nucleic acid multifunctional sequence to the microparticle, (ii) binding the polypeptide to the polypeptide binding moiety; and (iii) binding the DNA encoded compound to the polypeptide.

In aspects, step (i) comprises covalently conjugating the multifunctional sequence to the microparticle. In aspects, step (i) comprises covalently conjugating a polypeptide binding moiety to the microparticle. In aspects, the conjugation is a chemical conjugation. In aspects, step (i) comprises non-covalently conjugating the multifunctional sequence to the microparticle. In aspects, step (i) comprises non-covalently conjugating a polypeptide binding moiety to the microparticle.

In aspects, the microparticle comprises one or more functional reactive groups. In aspects, the functional reactive group is a bioconjugate reactive group. In aspects, the functional reactive group is an amine group or a carboxy group, hydroxyl, sulfhydryl, aldehyde, or any functional group used in organic synthesis.

In aspects, the functional reactive group is functionalized with a dibenzylcyclooctyne (DBCO). In aspects, the method further comprises reacting the DBCO with a nucleic acid multifunctional sequence azide. In aspects, the method further comprises reacting the DBCO with a polypeptide binding moiety azide.

In aspects, the method further comprises encapsulating the composition.

In aspects, the method further comprises producing the polypeptide in situ by transcribing and translating the polypeptide encoding nucleic acid sequence. In aspects, the polypeptide is produced by in-vitro transcription.

In aspects, a method of making a library composition of the disclosure is provided, the method comprising combining a plurality of microparticles, a plurality of polypeptide binding moieties, a plurality of polypeptides, a plurality of nucleic acid multifunctional sequences and a plurality of DNA encoded compounds under conditions sufficient for (i) attaching the polypeptide binding moieties and the nucleic acid multifunctional sequences to the microparticles, (ii) binding the polypeptides to the polypeptide binding moieties; and (iii) binding the DNA encoded compounds to the polypeptides.

In aspects, the steps (i), (ii), and (iii) are performed individually for each polypeptide of the plurality of polypeptides. In aspects, the steps (i), (ii), and (iii) are performed individually for each nucleic acid multifunctional sequence of the plurality of nucleic acid multifunctional sequences. In aspects, the steps (i), (ii), and (iii) are performed individually for each DNA encoded compound of the plurality of DNA encoded compounds. In aspects, the individually produced compositions are combined into one library composition.

### IV. Methods of Using Compositions

Provided herein are, inter alia, methods of using the compositions and libraries of compositions described herein.

In one aspect, a method of screening for one or more DNA encoded compounds that bind to a target polypeptide is provided, the method comprising: (i) contacting a plurality of DNA encoded compounds with a target polypeptide bound to a microparticle under conditions that allow for binding of the plurality of DNA encoded compounds to the target polypeptide, wherein the microparticle is bound to a nucleic acid multifunctional sequence, wherein said nucleic acid multifunctional sequence comprises: a nucleic acid polypeptide identifier sequence; and a first hybridization sequence; wherein each DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, (ii) removing DNA encoded compounds not binding to the polypeptide; (iii) compartmentalizing each microparticle; (iv) amplifying the DNA compound sequence to produce a complement of the DNA compound sequence, wherein the complement of the DNA compound sequence comprises a complement of the second hybridization sequence; (v) hybridizing the complement of the second hybridization sequence to the first hybridization sequence; and (vi) elongating the nucleic acid multifunctional sequence with a polymerase to produce a nucleic acid identification sequence comprising the nucleic acid compound identifier sequence and the nucleic acid polypeptide identifier sequence, thereby identifying the one or more DNA encoded compounds that bind to a target polypeptide.

In an aspect, a method of screening for one or more DNA encoded compounds that bind to a target polypeptide is provided, the method comprising: (i) contacting a plurality of DNA encoded compounds with a plurality of target polypeptides bound to a microparticle under conditions that allow for binding of the plurality of DNA encoded compounds to the polypeptides, wherein the plurality of target polypeptides bound to a microparticle comprises a nucleic acid multifunctional sequence bound to said microparticle, wherein said nucleic acid multifunctional sequence comprises: a nucleic acid polypeptide identifier sequence; and a first hybridization sequence; wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein the complement of the second hybridization sequence is capable of hybridizing to said first hybridization sequence, (ii) removing DNA encoded compounds not binding to a target polypeptide; (iii) compartmentalizing each microparticle; and (iv) amplifying the DNA compound sequence to produce a complement of the DNA compound sequence, wherein the complement of the DNA compound sequence comprises a complement of the second hybridization sequence; (v) hybridizing the complement of the second hybridization sequence to the first hybridization sequence; and (vi) elongating the nucleic acid multifunctional sequence with a polymerase to produce a nucleic acid identification sequence comprising the nucleic acid compound identifier sequence and the nucleic acid polypeptide identifier, thereby identifying the one or more DNA encoded compounds that bind to a target polypeptide.

In an aspect, a method of screening for one or more DNA encoded compounds that bind to a target polypeptide is provided, the method comprising: (i) transcribing a target polypeptide in situ by transcribing and translating a target polypeptide encoding nucleic acid sequence on a microparticle, wherein the microparticle is bound to a nucleic acid multifunctional sequence; and a target polypeptide binding moiety wherein said nucleic acid a multifunctional sequence comprises: a nucleic acid polypeptide identifier sequence; the target polypeptide encoding nucleic acid sequence; and a first hybridization sequence; and (ii) binding the target polypeptide to the target polypeptide binding moiety under conditions sufficient for binding the target polypeptide to the target polypeptide binding moiety; (iii) contacting a plurality of DNA encoded compounds with the target polypeptide bound to the microparticle under conditions that allow for binding of the plurality of DNA encoded compounds to the target polypeptide, wherein each DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence; (iv) removing DNA encoded compounds not binding to the target polypeptide; (v) compartmentalizing each microparticle; (vi) amplifying the DNA compound sequence to produce a complement of the DNA compound sequence, wherein the complement of the DNA compound sequence comprises a complement of the second hybridization sequence; (vii) hybridizing the complement of the second hybridization sequence to the first hybridization sequence; and (viii) elongating the nucleic acid multifunctional sequence with a polymerase to produce a nucleic acid identification sequence comprising the nucleic acid compound identifier sequence and the nucleic acid polypeptide identifier sequence, thereby identifying the one or more DNA encoded compounds that bind to a target polypeptide.

In an aspect, a method of screening for one or more DNA encoded compounds that bind to a target polypeptide is provided, the method comprising: (i) transcribing a plurality of target polypeptides in situ by transcribing and translating a plurality of target polypeptide encoding nucleic acid sequences on a plurality of microparticles, wherein each of the microparticles of the plurality of microparticles are bound to a nucleic acid multifunctional sequence; and a target polypeptide binding moiety wherein said nucleic acid a multifunctional sequence comprises: a nucleic acid polypeptide identifier sequence; the target polypeptide encoding nucleic acid sequence; and a first hybridization sequence; and (ii) binding the plurality of target polypeptides to the target polypeptide binding moiety under conditions sufficient for binding the target polypeptide to the target polypeptide binding moiety; (iii) contacting a plurality of DNA encoded compounds with the plurality of target polypeptide bound to the plurality of microparticles under conditions that allow for binding of the plurality of DNA encoded compounds to the plurality of target polypeptides, wherein each DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence; (iv) removing DNA encoded compounds not binding to the target polypeptide; (v) compartmentalizing each microparticle; (vi) amplifying the DNA compound sequence to produce a complement of the DNA compound sequence, wherein the complement of the DNA compound sequence comprises a complement of the second hybridization sequence; (vii) hybridizing the complement of the second hybridization sequence to the first hybridization sequence; and (viii) elongating the nucleic acid multifunctional sequence with a polymerase to produce a nucleic acid identification sequence comprising the nucleic acid compound identifier sequence and the nucleic acid polypeptide identifier sequence, thereby identifying the one or more DNA encoded compounds that bind to a target polypeptide.

In some embodiments, the target polypeptides of the plurality of target peptides are the same polypeptide. In some embodiments, the target polypeptides of the plurality of target peptides are different polypeptides. In some embodiments, the target polypeptides of the plurality of target peptides bound to the same microparticle are the same polypeptide. In some embodiments, the target polypeptides of the plurality of target peptides bound to the same microparticle are different polypeptides.

In some embodiments, the target polypeptide is produced by in-vitro transcription. In some embodiments, the steps (i) and (ii) are performed individually for each target polypeptide of the plurality of target polypeptides. In some embodiments, the steps (i) and (ii), are performed individually for each nucleic acid multifunctional sequence of the plurality of nucleic acid multifunctional sequences. In some embodiments, the steps (i) and (ii), are performed individually for each microparticle of the plurality of microparticles.

In some embodiments, the method step (i) and/or (ii) further comprises encapsulating the microparticle.

In some embodiments, the method further comprises de-coding the compound from the corresponding nucleic acid compound identifier sequence.

In aspects, a method of screening for one or more DNA encoded compounds that bind to a target molecule is provided, the method comprising: contacting a plurality of DNA encoded compounds with a target molecule bound to a microparticle under conditions that allow the DNA encoded compounds to bind the target molecule, wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, wherein the microparticle comprises a nucleic acid multifunctional sequence bound to said microparticle, wherein said nucleic acid multifunctional sequence comprises: a nucleic acid target molecule identifier sequence; and a first hybridization sequence; removing DNA encoded compounds not binding to the target molecule; compartmentalizing each microparticle; and linking each target molecule bound to a DNA encoded compound to the DNA encoded compound by hybridizing the first and second hybridization sequences, thereby identifying the one or more DNA encoded compounds that bind to a target molecule by de-coding from corresponding nucleic acid compound identifier sequence.

In aspects, a method of screening for one or more DNA encoded compounds that bind to a target molecule is provided, the method comprising: contacting a plurality of DNA encoded compounds with a plurality of target molecules bound to a microparticle under conditions that allow the DNA encoded compounds to bind the target molecules, wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, wherein the plurality of target molecules bound to a microparticle comprises a nucleic acid multifunctional sequence bound to said microparticle, wherein said nucleic acid multifunctional sequence comprises: a nucleic acid target molecule identifier sequence; and a first hybridization sequence, removing DNA encoded compounds not binding to a target molecule; compartmentalizing each microparticle; and linking each target molecule bound to a DNA encoded compound to the DNA encoded compound by hybridizing the first and second hybridization sequences, thereby identifying the one or more DNA encoded compounds that bind to a target molecule by de-coding from corresponding nucleic acid compound identifier sequence.

In aspects, a method of screening for one or more DNA encoded compounds that bind to a polypeptide is provided, the method comprising: contacting a plurality of DNA encoded compounds with a polypeptide bound to a microparticle under conditions that allow the DNA encoded compounds to bind the polypeptide, wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, wherein the microparticle comprises a nucleic acid multifunctional sequence bound to said microparticle, wherein said nucleic acid multifunctional sequence comprises: a nucleic acid polypeptide identifier sequence; and a first hybridization sequence, removing DNA encoded compounds not binding to the polypeptide; compartmentalizing each microparticle; and linking each polypeptide bound to a DNA encoded compound to the DNA encoded compound by hybridizing the first and second hybridization sequences, thereby identifying the one or more DNA encoded compounds that bind to a polypeptide by de-coding from corresponding nucleic acid compound identifier sequence.

In aspects, a method of screening for one or more DNA encoded compounds that bind to a polypeptide is provided, the method comprising: contacting a plurality of DNA encoded compounds with a plurality of polypeptides bound to a microparticle under conditions that allow the DNA encoded compounds to bind the polypeptides, wherein a DNA encoded compound of the plurality of DNA encoded compounds comprises a compound bound to a DNA compound sequence, wherein the DNA compound sequence comprises: a nucleic acid compound identifier sequence; and a second hybridization sequence wherein a complement of the second hybridization sequence is capable of hybridizing to said first hybridization sequence, wherein the plurality of polypeptides bound to a microparticle comprises a nucleic acid multifunctional sequence bound to said microparticle, wherein said nucleic acid multifunctional sequence comprises: a nucleic acid polypeptide identifier sequence; and a first hybridization sequence, removing DNA encoded compounds not binding to a polypeptide; compartmentalizing each microparticle; and linking each polypeptide bound to a DNA encoded compound to the DNA encoded compound by hybridizing the first and second hybridization sequences, thereby identifying the one or more DNA encoded compounds that bind to a polypeptide by de-coding from corresponding nucleic acid compound identifier sequence.

In aspects, the nucleic acid multifunctional sequence comprises a polypeptide encoding nucleic acid.

In aspects, the method further comprises amplifying the DNA compound sequence. In aspects, the method further comprises amplifying the DNA compound sequence hybridized to the nucleic acid multifunctional sequence, wherein the amplification forms a nucleic acid identification sequence.

In aspects, the method comprises a plurality of target molecules. In aspects, the method comprises a plurality of polypeptides. In aspects, the polypeptides are the same polypeptide. In aspects, the polypeptides are different polypeptides. In aspects, the DNA encoded compounds are the same compound. In aspects, the DNA encoded compounds are different compounds.

In aspects, the microparticles are compartmentalized in an emulsion. In some embodiments, the emulsion is composed of discrete aqueous phase microdroplets enclosed by a thermostable oil phase. Each microdroplet can contains amplification reaction solution (i.e., the reagents necessary for nucleic acid amplification). In aspects, the microparticles are compartmentalized in a vessel. Following amplification of the nucleic acid template, the emulsion is “broken” (also referred to as “demulsification”). Amplification products can be purified and used as template for further amplification reactions, for example for a preparation to next generation sequencing (NGS). In embodiments, a compartment used to encapsulate or compartmentalize a composition (e.g., a library or microparticle as disclosed herein) is an aqueous droplet within an emulsion, as described herein.

In aspects, the method further comprises identifying the one or more target molecules that specifically bind to a DNA encoded compound by de-coding from corresponding nucleic acid polypeptide identifier nucleic acid sequences.

In aspects, the method further comprises identifying the one or more polypeptides that specifically bind to a DNA encoded compound by de-coding from corresponding nucleic acid polypeptide identifier nucleic acid sequences.

In aspects, the method further comprises amplifying a nucleic acid identification sequence comprising the nucleic acid polypeptide identifier sequence, the first hybridization sequence, and the nucleic acid compound identifier sequence.

In aspects, the method further comprises sequencing the nucleic acid identification sequence.

In aspects, the method further comprises identifying and determining unique identifier sequencing events.

In aspects, the method further comprises determining the number of target molecule binding events for each unique nucleic acid compound identifier sequence.

In aspects, the method further comprises determining a target molecule binding frequency for each DNA encoded compound.

In aspects, the method further comprises summing all target molecule binding events across two or more microparticles comprising the same polypeptide.

In aspects, the method further comprises determining a distribution of target molecule events.

In aspects, the method further comprises determining a distribution of target molecule binding events over 2 or more repeats.

In aspects, the disclosure provides for a computer-implemented method for identifying a compound having an affinity with a target molecule, the method comprising querying a machine learning engine for a proposed compound having an affinity with the target molecule, wherein the machine learning engine was trained using the target molecule binding events of the disclosure; and receiving the compound from the machine learning engine.

In aspects, the method further comprises determining the number of polypeptide binding events for each unique nucleic acid compound identifier sequence.

In aspects, the method further comprises determining a polypeptide binding frequency for each DNA encoded compound.

In aspects, the method further comprises summing all polypeptide binding events across two or more microparticles comprising the same polypeptide.

In aspects, the method further comprises determining a distribution of polypeptide binding events.

In aspects, the method steps are repeated. In aspects, the method steps are repeated once. In aspects, the method steps are repeated two times. In aspects, the method steps are repeated at least three times.

In aspects, the method further comprises determining a distribution of polypeptide binding events over 2 or more repeats.

In aspects, the disclosure provides for a computer-implemented method for identifying a compound having an affinity with a polypeptide, the method comprising querying a machine learning engine for a proposed compound having an affinity with the polypeptide, wherein the machine learning engine was trained using the polypeptide binding events of the disclosure; and receiving the compound from the machine learning engine.

### Embodiments

Embodiment 1. A composition comprising
   (i) a solid microparticle;
   (ii) a polypeptide bound to said solid microparticle;
   (iii) a nucleic acid multifunctional sequence bound to said microparticle, wherein said nucleic acid multifunctional sequence comprises:
      a nucleic acid polypeptide identifier sequence;
      a polypeptide encoding nucleic acid sequence; and
      a first hybridization sequence; and
   (iv) a DNA encoded compound bound to said polypeptide, said DNA encoded compound comprising a compound bound to a DNA compound sequence, said DNA compound sequence comprising:
      a nucleic acid compound identifier sequence; and
      a second hybridization sequence capable of hybridizing to said first hybridization sequence.
Embodiment 2. A composition comprising
   (i) a solid microparticle;
   (ii) a polypeptide bound to said solid microparticle;
   (iii) a nucleic acid multifunctional sequence bound to said microparticle, wherein said nucleic acid multifunctional sequence comprises:
      a nucleic acid polypeptide identifier sequence;
      a polypeptide encoding nucleic acid sequence;
      a first hybridization sequence; and
   (iv) a DNA encoded compound bound to said nucleic acid multifunctional sequence, said DNA encoded compound comprising a compound bound to a DNA compound sequence, said DNA compound sequence comprising:
      a nucleic acid compound identifier sequence; and
      a second hybridization sequence, wherein the second hybridization sequence and the first hybridization sequence are bound to each other.
Embodiment 3. A composition comprising
   (i) a solid microparticle;
   (ii) a polypeptide bound to said solid microparticle;
   (iii) a nucleic acid multifunctional sequence bound to said microparticle, wherein said nucleic acid multifunctional sequence comprises:
      a nucleic acid polypeptide identifier sequence;
      a polypeptide encoding nucleic acid sequence; and
      a first hybridization sequence; and
   (iv) a DNA encoded compound, said DNA encoded compound comprising a compound bound to a DNA compound sequence, said DNA compound sequence comprising:
      a nucleic acid compound identifier sequence; and
      a second hybridization sequence capable of hybridizing to said first hybridization sequence;
   wherein (i) to (iv) are encapsulated in the same compartment.
Embodiment 4. The composition of embodiment 3, further comprising a nucleic acid identification sequence comprising the nucleic acid polypeptide identifier sequence, the first hybridization sequence, and the nucleic acid compound identifier sequence.
Embodiment 5. The composition of any one of embodiments 1 to 4, wherein the microparticle is at least 1 μm.
Embodiment 6. The composition of any one of embodiments 1 to 4, wherein the microparticle is at most 500 μm.
Embodiment 7. The composition of any one of embodiments 1 to 6, wherein the microparticle is a bead.
Embodiment 8. The composition of any one of embodiments 1 to 7, wherein the polypeptide further comprises a protein tag.
Embodiment 9. The composition of embodiment 8, wherein the protein tag is selected from the group of Polyhistidine (His-tag), Glutathione S-transferase (GST), FLAG tag, SPY tag, calmodulin binding domain (CBD), chitin binding domain (CBD), choline-binding domain (CBD), albumin-binding protein (ABP), Bacteriophage T7 epitope (T7-tag), bacteriophage V5 epitope (V5-tag), biotin-carboxy carrier protein (BCCP), bluetongue virus tag (B-tag), chloramphenicol acetyl transferase (CAT), cellulose binding domain (CBP), E2 epitope, galactose-binding protein (GBP), green fluorescent protein (GFP), Glu-Glu (EE-tag), human influenza hemagglutinin (HA), HaloTag, maltose-binding protein (MBP), streptavadin-binding peptide (SBP), Myc epitope tag, strep-tag, and biotin.
Embodiment 10. The composition of any one of embodiments 1 to 9, wherein the polypeptide is bound non-covalently to the microparticle.
Embodiment 11. The composition of any one of embodiments 1 to 9, wherein the polypeptide is bound covalently to the microparticle.
Embodiment 12. The composition of any one of embodiments 1 to 11, wherein the polypeptide is bound to the microparticle by a polypeptide binding moiety.
Embodiment 13. The composition of any one of embodiments 1 to 12, wherein the polypeptide binding moiety is selected from the group of a divalent metal, glutathione, anti-FLAG antibody or fragment thereof, calmodulin, chitin, choline, anti-E2 antibody or a fragment thereof, cellulose, albumin, streptavidin, anti-bluetongue virus antibody or a fragment thereof, galactose, anti-GFP antibody or a fragment thereof, biotin, polypeptide specific antibody, and maltose.
Embodiment 14. The composition of any one of embodiments 12 to 14, wherein the polypeptide binding moiety further comprises a linker.
Embodiment 15. The composition of embodiment 14, wherein the linker is a polypeptide linker, a chemical linker, or a nucleic acid linker.
Embodiment 16. The composition of any one of embodiment 1 to 15, wherein the composition is encapsulated.
Embodiment 17. The composition of any one of embodiment 1 to 16, wherein the composition is partitioned into a compartment.
Embodiment 18. The composition of embodiment 17, wherein the compartment is an emulsion.
Embodiment 19. The composition of embodiment 17, wherein the emulsion is an oil in water emulsion.
Embodiment 20. The composition of embodiment 17, wherein the size of the compartment is between 10 μm and 1 mm.
Embodiment 21. The composition of any one of embodiments 1 to 20, wherein the compound is selected from the group of a small molecule, a macromolecule, a peptide, a protein, a nucleic acid, a lipid, a glycan and a cell.
Embodiment 22. The composition of embodiment 21, wherein the protein is a protein complex.
Embodiment 23. The composition of embodiment 21, wherein the peptide is a neoantigen peptide.
Embodiment 24. The composition of embodiment 21, wherein the nucleic acid is a RNA, a DNA, or a mixture of RNA or DNA.
Embodiment 25. The composition of embodiment 21, wherein the nucleic acid is an aptamer.
Embodiment 26. The composition of any one of embodiments 1 to 26, wherein the nucleic acid multifunctional sequence is bound to the microparticle by a nucleic acid linker or a chemical linker.
Embodiment 27. The composition of any one of embodiments 1 to 26, wherein the nucleic acid multifunctional sequence further comprises a unique identifier.
Embodiment 28. The composition of any one of embodiments 1 to 27, wherein the nucleic acid multifunctional sequence is a DNA, RNA, or a mixture of DNA and RNA.
Embodiment 29. The composition of any one of embodiments 1 to 28, wherein the nucleic acid multifunctional sequence is a single stranded nucleic acid.
Embodiment 30. The composition of any one of embodiments 1 to 28, wherein the nucleic acid multifunctional sequence is a double stranded nucleic acid.
Embodiment 31. The composition of embodiment 30, wherein the nucleic acid multifunctional sequence is a double stranded nucleic acid and the 5′ and 3′ ends each comprise a hairpin of unpaired bases and the hairpin connects the 5′ end of one strand to the 3′ end of the other strand.
Embodiment 32. The composition of embodiment 31, wherein the hairpins are at least 10 bases long.
Embodiment 33. The composition of any one of embodiments 1 to 32, wherein the nucleic acid multifunctional sequence further comprises a first amplification sequence.
Embodiment 34. The composition of any one of embodiments 1 to 32, wherein the DNA compound sequence further comprises a second amplification sequence.
Embodiment 35. The composition of any one of embodiments 1 to 34, wherein the nucleic acid multifunctional sequence is resistant to nuclease degradation.
Embodiment 36. The composition of any one of embodiments 1 to 35, wherein the polypeptide encoding nucleic acid sequence encodes all or a part of the polypeptide.
Embodiment 37. The composition of any one of embodiments 1 to 36, wherein about 10⁵ to 10⁶ of said nucleic acid multifunctional sequences are attached to the microparticle.
Embodiment 38. The composition of any one of embodiments 1 to 37, wherein about 10⁶ to 10⁹ of said polypeptides are attached to the microparticle.
Embodiment 39. A method of making a composition of any one of embodiments 1-38, the method comprising combining the microparticle, a polypeptide binding moiety, the polypeptide, the nucleic acid multifunctional sequence and the DNA encoded compound under conditions sufficient for (i) attaching the polypeptide binding moiety and the nucleic acid multifunctional sequence to the microparticle, (ii) binding the polypeptide to the polypeptide binding moiety; and (iii) binding the DNA encoded compound to the polypeptide.
Embodiment 40. The method of embodiment 39, wherein step (i) comprises covalently conjugating the multifunctional sequence to the microparticle.
Embodiment 41. The method of any one of embodiments 39 or 40, wherein step (i) comprises covalently conjugating a polypeptide binding moiety to the microparticle.
Embodiment 42. The method of embodiment 41, wherein the conjugation is a chemical conjugation.
Embodiment 43. The method of any one of embodiments 39 to 42, wherein the microparticle comprises one or more functional reactive groups.
Embodiment 44. The method of embodiment 43, wherein the functional reactive group is an amine group or, a carboxy group.
Embodiment 45. The method of embodiment 44, wherein the functional reactive group is functionalized with a dibenzylcyclooctyne (DBCO).
Embodiment 46. The method of embodiment 45, further comprising, reacting the DBCO with a nucleic acid multifunctional sequence azide.
Embodiment 47. The method of embodiment 45, further comprising, reacting the DBCO with a polypeptide binding moiety azide.
Embodiment 48. The method of any one of embodiments 39 to 47, further comprising encapsulating the composition.
Embodiment 49. The method of any one of embodiments 39 to 48, further comprising producing the polypeptide in situ by transcribing and translating the polypeptide encoding nucleic acid sequence.
Embodiment 50. A library, wherein the library comprises a plurality of compositions of any one of embodiments 1 to 38, or a plurality of compositions produced by the method of any one of embodiments 39 to 49.
Embodiment 51. The library of embodiment 50, wherein the library comprises a plurality of polypeptides.
Embodiment 52. The library of embodiment 51, wherein the polypeptides are the same polypeptide.
Embodiment 53. The library of embodiment 51, wherein the polypeptides are different polypeptides.
Embodiment 54. The library of any one of embodiments 50 to 53, wherein the library comprises a plurality of DNA encoded compounds.
Embodiment 55. The library of embodiment 54, wherein the DNA encoded compounds are the same compound.
Embodiment 56. The library of embodiment 54, wherein the DNA encoded compounds are different compounds.
Embodiment 57. The library of any one of embodiments 50 to 56, wherein the library is in an emulsion.
Embodiment 58. The library of any one of embodiments 50 to 56, wherein each member of the library is encapsulated.
Embodiment 59. The library of any one of embodiments 50 to 56, wherein each member of the library is partitioned into a compartment.
Embodiment 60. The library of embodiment 59, wherein the compartment is an emulsion.
Embodiment 61. The library of embodiment 60, wherein the emulsion is an oil in water emulsion.
Embodiment 62. The library of any one of embodiments 50 to 61, wherein the library is in a vessel.
Embodiment 63. The library of any one of embodiments 50 to 61, wherein each member of the library is in a vessel.
Embodiment 64. The library of any one of embodiments 50 to 63, wherein the polypeptide is derived from a transcriptome.
Embodiment 65. A method of screening for one or more DNA encoded compounds that bind to a polypeptide, the method comprising:
   (i) contacting a plurality of DNA encoded compounds with a polypeptide bound to a solid microparticle under conditions that allow the DNA encoded compounds to bind the polypeptide, wherein a DNA encoded compound of the plurality of DNA encoded compounds comprises a compound bound to a DNA compound sequence,
      wherein the DNA compound sequence comprises:
         a nucleic acid compound identifier sequence; and
         a second hybridization sequence capable of hybridizing to said first hybridization sequence,
      wherein the solid microparticle comprises a nucleic acid multifunctional sequence bound to said microparticle, wherein said nucleic acid multifunctional sequence comprises:
         a nucleic acid polypeptide identifier sequence;
         a polypeptide encoding nucleic acid sequence; and
         a first hybridization sequence,
   (ii) removing DNA encoded compounds not binding to the polypeptide;
   (iii) compartmentalizing each microparticle; and
   (iv) linking each polypeptide bound to a DNA encoded compound to the DNA encoded compound by hybridizing the first and second hybridization sequences, thereby identifying the one or more DNA encoded compounds that bind to a polypeptide by de-coding from corresponding nucleic acid compound identifier sequence.
Embodiment 66. A method of screening for one or more DNA encoded compounds that bind to a polypeptide, the method comprising:
   (i) contacting a plurality of DNA encoded compounds with a plurality of polypeptides bound to a solid microparticle under conditions that allow the DNA encoded compounds to bind the polypeptides, wherein a DNA encoded compound of the plurality of DNA encoded compounds comprises a compound bound to a DNA compound sequence, wherein the DNA compound sequence comprises:
      a nucleic acid compound identifier sequence; and
      a second hybridization sequence capable of hybridizing to said first hybridization sequence,
      wherein the plurality of polypeptides bound to a solid microparticle comprises a nucleic acid multifunctional sequence bound to said microparticle, wherein said nucleic acid multifunctional sequence comprises:
      a nucleic acid polypeptide identifier sequence;
      a polypeptide encoding nucleic acid sequence; and
      a first hybridization sequence,
   (ii) removing DNA encoded compounds not binding to a polypeptide;
   (iii) compartmentalizing each microparticle; and
   (iv) linking each polypeptide bound to a DNA encoded compound to the DNA encoded compound by hybridizing the first and second hybridization sequences, thereby identifying the one or more DNA encoded compounds that bind to a polypeptide by de-coding from corresponding nucleic acid compound identifier sequence.
Embodiment 67. The method of embodiment 65 or 66, wherein the library comprises a plurality of polypeptides.
Embodiment 68. The method of embodiment 67, wherein the polypeptides are the same polypeptide.
Embodiment 69. The method of embodiment 67, wherein the polypeptides are different polypeptides.
Embodiment 70. The method of any one of embodiments 65 to 69, wherein the DNA encoded compounds are the same compound.
Embodiment 71. The method of any one of embodiments 65 to 69, wherein the DNA encoded compounds are different compounds.
Embodiment 72. The method of any one of embodiments 65 to 71, wherein the microparticles are compartmentalized in an emulsion.
Embodiment 73. The method of any one of embodiments 65 to 71, wherein the microparticles are compartmentalized in a vessel.
Embodiment 74. The method of any one of embodiments 65 to 73, the method further comprising identifying the one or more polypeptides that specifically bind to a DNA encoded compound by de-coding from corresponding nucleic acid polypeptide identifier nucleic acid sequences.
Embodiment 75. The method of any one of embodiments 65 to 74, further comprising amplifying a nucleic acid identification sequence comprising the nucleic acid polypeptide identifier sequence, the first hybridization sequence, and the nucleic acid compound identifier sequence.
Embodiment 76. The method of embodiment 75, further comprising sequencing the nucleic acid identification sequence.
Embodiment 77. The method of any one of embodiments 65 to 76, further comprising identifying and determining unique identifier sequencing events.
Embodiment 78. The method of embodiment 77, further comprising determining the number of polypeptide binding events for each unique nucleic acid compound identifier sequence.
Embodiment 79. The method of embodiment 78, further comprising determining a polypeptide binding frequency for each DNA encoded compound of the plurality of DNA encoded compounds.
Embodiment 80. The method of embodiment 79, further comprising summing all polypeptide binding events across two or more microparticles comprising the same polypeptide.
Embodiment 81. The method of embodiment 80, further comprising determining a distribution of polypeptide binding events.
Embodiment 82. The method of any one of embodiments 65 to 81, wherein the method steps are repeated.
Embodiment 83. The method of embodiment 82, further comprising determining a distribution of polypeptide binding events over 2 or more repeats.
Embodiment 84. A computer-implemented method for identifying a compound having an affinity with a polypeptide, the method comprising querying a machine learning engine for a proposed compound having an affinity with the polypeptide, wherein the machine learning engine was trained using the polypeptide binding events of any one of embodiments 65 to 82; and receiving the compound from the machine learning engine.
Embodiment 85. A method of screening for one or more DNA encoded compounds that bind to a target polypeptide, the method comprising:
   (i) contacting a plurality of DNA encoded compounds with a target polypeptide bound to a microparticle under conditions that allow for binding of the plurality of DNA encoded compounds to the target polypeptide, wherein the microparticle is bound to a nucleic acid multifunctional sequence, wherein said nucleic acid multifunctional sequence comprises:
      a nucleic acid polypeptide identifier sequence; and
      a first hybridization sequence;
      wherein each DNA encoded compound of the plurality of DNA encoded compounds comprises a compound bound to a DNA compound sequence, wherein the DNA compound sequence comprises:
      a nucleic acid compound identifier sequence; and
      a second hybridization sequence wherein a complement of the second hybridization sequence is capable of hybridizing to said first hybridization sequence,
   (ii) removing DNA encoded compounds not binding to the polypeptide;
   (iii) compartmentalizing each microparticle;
   (iv) amplifying the DNA compound sequence to produce a complement of the DNA compound sequence, wherein the complement of the DNA compound sequence comprises a complement of the second hybridization sequence;
   (v) hybridizing the complement of the second hybridization sequence to the first hybridization sequence; and
   (vi) elongating the nucleic acid multifunctional sequence with a polymerase to produce a nucleic acid identification sequence comprising the nucleic acid compound identifier sequence and the nucleic acid polypeptide identifier sequence,
      thereby identifying the one or more DNA encoded compounds that bind to a target polypeptide. by de-coding from the corresponding nucleic acid compound identifier sequence.
Embodiment 86. A method of screening for one or more DNA encoded compounds that bind to a target polypeptide, the method comprising:
   (i) contacting a plurality of DNA encoded compounds with a plurality of target polypeptides bound to a microparticle under conditions that allow for binding of the plurality of DNA encoded compounds to the polypeptides, wherein the plurality of target polypeptides bound to a microparticle comprises a nucleic acid multifunctional sequence bound to said microparticle, wherein said nucleic acid multifunctional sequence comprises:
      a nucleic acid polypeptide identifier sequence; and
      a first hybridization sequence;
      wherein a DNA encoded compound of the plurality of DNA encoded compounds comprises a compound bound to a DNA compound sequence, wherein the DNA compound sequence comprises:
      a nucleic acid compound identifier sequence; and
         a second hybridization sequence wherein the complement of the second hybridization sequence is capable of hybridizing to said first hybridization sequence,
   (ii) removing DNA encoded compounds not binding to a target polypeptide;
   (iii) compartmentalizing each microparticle; and
   (iv) amplifying the DNA compound sequence to produce a complement of the DNA compound sequence, wherein the complement of the DNA compound sequence comprises a complement of the second hybridization sequence;
   (v) hybridizing the complement of the second hybridization sequence to the first hybridization sequence; and
   (vi) elongating the nucleic acid multifunctional sequence with a polymerase to produce a nucleic acid identification sequence comprising the nucleic acid compound identifier sequence and the nucleic acid polypeptide identifier,
      thereby identifying the one or more DNA encoded compounds that bind to a target polypeptide by de-coding from corresponding nucleic acid compound identifier sequence.
Embodiment 87. The method of any one of embodiments 85 or 86, wherein the method further comprises de-coding the compound from the corresponding nucleic acid compound identifier sequence.
Embodiment 88. The method of any one of embodiments 85 to 87, wherein the method comprises a plurality of target polypeptides.
Embodiment 89. The method of embodiment 88, wherein the target polypeptides are the same target polypeptide.
Embodiment 90. The method of embodiment 88, wherein the target polypeptides are different target polypeptides.
Embodiment 91. The method of any one of embodiments 85 to 90, wherein the DNA encoded compounds are the same compound.
Embodiment 92. The method of any one of embodiments 85 to 91, wherein the DNA encoded compounds are different compounds.
Embodiment 93. The method of any one of embodiments 85 to 92, wherein the microparticles are compartmentalized in an emulsion.
Embodiment 94. The method of any one of embodiments 85 to 93, wherein the microparticles are compartmentalized in a vessel.
Embodiment 95. The method of any one of embodiments 85 to 94, the method further comprising identifying the one or more target polypeptides that specifically bind to a DNA encoded compound by de-coding from corresponding nucleic acid polypeptide identifier nucleic acid sequences.
Embodiment 96. The method of any one of embodiments 85 to 95, further comprising amplifying the nucleic acid identification sequence comprising the nucleic acid polypeptide identifier sequence, the first hybridization sequence, and the nucleic acid compound identifier sequence.
Embodiment 97. The method of embodiment 96, further comprising sequencing the nucleic acid identification sequence.
Embodiment 98. The method of any one of embodiments 85 to 97, further comprising identifying and determining unique identifier sequencing events.
Embodiment 99. The method of embodiment 98, further comprising determining the number of target polypeptide binding events for each unique nucleic acid compound identifier sequence.
Embodiment 100. The method of embodiment 99, further comprising determining a target polypeptide binding frequency for each DNA encoded compound of the plurality of DNA encoded compounds.
Embodiment 101. The method of embodiment 100, further comprising summing all target polypeptide binding events across two or more microparticles comprising the same target polypeptide.
Embodiment 102. The method of embodiment 101, further comprising determining a distribution of target polypeptide binding events.
Embodiment 103. The method of any one of embodiments 85 to 102, wherein the method steps are repeated.
Embodiment 104. The method of embodiment 103, further comprising determining a distribution of target polypeptide binding events over 2 or more repeats.
Embodiment 105. A computer-implemented method for identifying a compound having an affinity with a target polypeptide, the method comprising
   querying a machine learning engine for a proposed compound having an affinity with the
   target polypeptide, wherein the machine learning engine was trained using the target polypeptide binding events of any one of embodiments 85 to 104; and
   receiving the compound from the machine learning engine.
Embodiment 106. A composition comprising
   (i) a microparticle;
   (ii) a target molecule bound to said microparticle; and
   (iii) a nucleic acid multifunctional sequence bound to said microparticle, wherein said nucleic acid multifunctional sequence comprises:
      a nucleic acid target molecule identifier sequence; and
      a first hybridization sequence.
Embodiment 107. A composition comprising
   (i) a microparticle;
   (ii) a target molecule bound to said microparticle;
   (iii) a nucleic acid multifunctional sequence bound to said microparticle, wherein said nucleic acid multifunctional sequence comprises:
      a nucleic acid target molecule identifier sequence; and
      a first hybridization sequence; and
   (iv) a DNA encoded compound bound to said target molecule, said DNA encoded compound comprising a compound bound to a DNA compound sequence, said DNA compound sequence comprising:
      a nucleic acid compound identifier sequence; and
      a second hybridization sequence wherein a complement of the second hybridization sequence is capable of hybridizing to said first hybridization sequence.
Embodiment 108. A composition comprising
   (i) a microparticle;
   (ii) a target molecule bound to said microparticle;
   (iii) a nucleic acid multifunctional sequence bound to said microparticle, wherein said nucleic acid multifunctional sequence comprises:
      a nucleic acid target molecule identifier sequence;
      a first hybridization sequence; and
   (iv) a DNA encoded compound bound to said nucleic acid multifunctional sequence, said DNA encoded compound comprising a compound bound to a DNA compound sequence, said DNA compound sequence comprising:
      a nucleic acid compound identifier sequence; and
      a complement of a second hybridization sequence, wherein the complement of the second hybridization sequence and the first hybridization sequence are bound to each other.
Embodiment 109. The composition for any one of embodiments 106 to 108, wherein the target molecule is a target polypeptide.
Embodiment 110. A composition comprising
   (i) a microparticle;
   (ii) a target polypeptide bound to said microparticle;
   (iii) a nucleic acid multifunctional sequence bound to said microparticle, wherein said nucleic acid multifunctional sequence comprises:
      a nucleic acid polypeptide identifier sequence; and
      a first hybridization sequence; and
   (iv) a DNA encoded compound bound to said target polypeptide, said DNA encoded compound comprising a compound bound to a DNA compound sequence, said DNA compound sequence comprising:
      a nucleic acid compound identifier sequence; and
      a second hybridization sequence wherein a complement of the second hybridization sequence is capable of hybridizing to said first hybridization sequence.
Embodiment 111. A composition comprising
   (i) a microparticle;
   (ii) a target polypeptide bound to said microparticle;
   (iii) a nucleic acid multifunctional sequence bound to said microparticle, wherein said nucleic acid multifunctional sequence comprises:
      a nucleic acid polypeptide identifier sequence;
      a first hybridization sequence; and
   (iv) a DNA encoded compound bound to said nucleic acid multifunctional sequence, said DNA encoded compound comprising a compound bound to a DNA compound sequence, said DNA compound sequence comprising:
      a nucleic acid compound identifier sequence; and
      a complement of a second hybridization sequence, wherein the complement of the second hybridization sequence and the first hybridization sequence are bound to each other.
Embodiment 112. The composition of any one of embodiments 106 to 111, wherein the nucleic acid multifunctional sequence further comprises a target polypeptide encoding nucleic acid sequence.
Embodiment 113. The composition of any one of embodiments 106 to 112, further comprising a nucleic acid identification sequence comprising the nucleic acid target molecule identifier sequence or nucleic acid polypeptide identifier sequence, the first hybridization sequence, and the nucleic acid compound identifier sequence.
Embodiment 114. The composition of any one of embodiments 106 to 113, wherein the microparticle is a hydrogel.
Embodiment 115. The composition of any one of embodiments 106 to 114, wherein the longest dimension of the microparticle is at least 1 μm.
Embodiment 116. The composition of any one of embodiments 106 to 114, wherein the longest dimension of the microparticle is at most 500 μm.
Embodiment 117. The composition of any one of embodiments 106 to 114, wherein the microparticle is a bead.
Embodiment 118. The composition of any one of embodiments 106 to 115, wherein the target polypeptide further comprises a protein tag.
Embodiment 119. The composition of embodiment 118, wherein the protein tag is selected from the group of Polyhistidine (His-tag), Glutathione S-transferase (GST), FLAG tag, SPY tag, calmodulin binding domain (CBD), chitin binding domain (CBD), choline-binding domain (CBD), albumin-binding protein (ABP), Bacteriophage T7 epitope (T7-tag), bacteriophage V5 epitope (V5-tag), biotin-carboxy carrier protein (BCCP), bluetongue virus tag (B-tag), chloramphenicol acetyl transferase (CAT), cellulose binding domain (CBP), E2 epitope, galactose-binding protein (GBP), green fluorescent protein (GFP), Glu-Glu (EE-tag), human influenza hemagglutinin (HA), HaloTag, maltose-binding protein (MBP), streptavadin-binding peptide (SBP), Myc epitope tag, strep-tag, and biotin.
Embodiment 120. The composition of any one of embodiments 106 to 119, wherein the target polypeptide is bound non-covalently to the microparticle.
Embodiment 121. The composition of any one of embodiments 106 to 120, wherein the target polypeptide is bound covalently to the microparticle.
Embodiment 122. The composition of any one of embodiments 106 to 120, wherein the target polypeptide is bound to the microparticle by a target polypeptide binding moiety.
Embodiment 123. The composition of embodiment 112, wherein the target polypeptide binding moiety is selected from the group of a divalent metal, glutathione, anti-FLAG antibody or fragment thereof, calmodulin, chitin, choline, anti-E2 antibody or a fragment thereof, cellulose, albumin, streptavidin, anti-bluetongue virus antibody or a fragment thereof, galactose, anti-GFP antibody or a fragment thereof, biotin, polypeptide specific antibody, and maltose.
Embodiment 124. The composition of any one of embodiments 118 to 123, wherein the target polypeptide binding moiety further comprises a linker.
Embodiment 125. The composition of embodiment 124, wherein the linker is a polypeptide linker, a chemical linker, or a nucleic acid linker.
Embodiment 126. The composition of any one of embodiment 106 to 125, wherein the composition is encapsulated.
Embodiment 127. The composition of any one of embodiment 106 to 126, wherein the composition is partitioned into a compartment.
Embodiment 128. The composition of embodiment 127, wherein the compartment is an aqueous droplet within an emulsion.
Embodiment 129. The composition of embodiment 128, wherein the emulsion is an oil in water emulsion.
Embodiment 130. The composition of embodiment 127, wherein the longest dimension of the compartment is between 10 μm and 1 mm.
Embodiment 131. The composition of any one of embodiments 106 to 130, wherein the compound is selected from the group of a small molecule, a macromolecule, a peptide, a polypeptide, a protein, a protein complex, a nucleic acid, a lipid, a carbohydrate, a glycan, and a cell.
Embodiment 132. The composition of embodiment 131, wherein the compound is a protein complex.
Embodiment 133. The composition of embodiment 131, wherein the compound is a neoantigen peptide.
Embodiment 134. The composition of embodiment 131, wherein the compound is a RNA, a DNA, or a mixture of RNA or DNA.
Embodiment 135. The composition of embodiment 131, wherein the compound is an aptamer.
Embodiment 136. The composition of any one of embodiments 106 to 135, wherein the nucleic acid multifunctional sequence is bound to the microparticle by a nucleic acid linker or a chemical linker.
Embodiment 137. The composition of any one of embodiments 106 to 136, wherein the nucleic acid multifunctional sequence further comprises a unique identifier.
Embodiment 138. The composition of any one of embodiments 106 to 137, wherein the nucleic acid multifunctional sequence is a DNA, RNA, or a mixture of DNA and RNA.
Embodiment 139. The composition of any one of embodiments 106 to 138, wherein the nucleic acid multifunctional sequence is a single stranded nucleic acid.
Embodiment 140. The composition of any one of embodiments 106 to 139, wherein the nucleic acid multifunctional sequence further comprises a first amplification sequence.
Embodiment 141. The composition of any one of embodiments 106 to 140, wherein the DNA compound sequence further comprises a second amplification sequence.
Embodiment 142. The composition of any one of embodiments 106 to 141, wherein the nucleic acid multifunctional sequence is resistant to nuclease degradation.
Embodiment 143. The composition of any one of embodiments 106 to 142, wherein about 10⁵ to 106 of said nucleic acid multifunctional sequences are attached to the microparticle.
Embodiment 144. The composition of any one of embodiments 106 to 143, wherein about 106 to 10⁹ of said target polypeptides are attached to the microparticle.
Embodiment 145. A method of making a composition of any one of embodiments 106 to 144, the method comprising combining the microparticle, a target polypeptide binding moiety, the target polypeptide, the nucleic acid multifunctional sequence under conditions sufficient for (i) attaching the target polypeptide binding moiety and the nucleic acid multifunctional sequence to the microparticle, and (ii) binding the target polypeptide to the target polypeptide binding moiety.
Embodiment 146. A method of making a composition of any one of embodiments 106-144, the method comprising combining the microparticle, a target polypeptide binding moiety, the target polypeptide, the nucleic acid multifunctional sequence and the DNA encoded compound under conditions sufficient for (i) attaching the target polypeptide binding moiety and the nucleic acid multifunctional sequence to the microparticle, (ii) binding the target polypeptide to the target polypeptide binding moiety; and (iii) binding the DNA encoded compound to the target polypeptide.
Embodiment 147. The method of embodiment 145 or 146, wherein step (i) comprises covalently conjugating the multifunctional sequence to the microparticle.
Embodiment 148. The method of any one of embodiments 145 or 146, wherein step (i) comprises covalently conjugating a target polypeptide binding moiety to the microparticle.
Embodiment 149. The method of embodiment 147 or 148, wherein the conjugation is a chemical conjugation.
Embodiment 150. The method of any one of embodiments 145 to 149, wherein the microparticle comprises one or more functional reactive groups.
Embodiment 151. The method of embodiment 150, wherein the functional reactive group is an amine group or, a carboxy group.
Embodiment 152. The method of embodiment 151, wherein the functional reactive group is functionalized with a dibenzylcyclooctyne (DBCO).
Embodiment 153. The method of embodiment 152, further comprising, reacting the DBCO with a nucleic acid multifunctional sequence azide.
Embodiment 154. The method of embodiment 152, further comprising, reacting the DBCO with a target polypeptide binding moiety azide.
Embodiment 155. The method of any one of embodiments 145 to 154, further comprising encapsulating the composition.
Embodiment 156. The method of any one of embodiments 145 to 155, further comprising producing the target polypeptide in situ by transcribing and translating the polypeptide encoding nucleic acid sequence.
Embodiment 157. A library, wherein the library comprises a plurality of compositions of any one of embodiments 106 to 144, or a plurality of compositions produced by the method of any one of embodiments 145 to 156.
Embodiment 158. The library of embodiment 157, wherein the library comprises a plurality of target polypeptides.
Embodiment 159. The library of embodiment 158, wherein the target polypeptides are the same target polypeptide.
Embodiment 160. The library of embodiment 158, wherein the target polypeptides are different target polypeptides.
Embodiment 161. The library of any one of embodiments 157 to 160, wherein the library comprises a plurality of DNA encoded compounds.
Embodiment 162. The library of embodiment 161, wherein the DNA encoded compounds are the same compound.
Embodiment 163. The library of embodiment 161, wherein the DNA encoded compounds are different compounds.
Embodiment 164. The library of any one of embodiments 158 to 163, wherein the library is in an emulsion.
Embodiment 165. The library of any one of embodiments 158 to 164, wherein each member of the library is encapsulated.
Embodiment 166. The library of any one of embodiments 158 to 165, wherein each member of the library is partitioned into a different compartment.
Embodiment 167. The library of embodiment 166, wherein each compartment is an aqueous droplet in an emulsion.
Embodiment 168. The library of embodiment 167, wherein the emulsion is an oil in water emulsion.
Embodiment 169. The library of embodiment 166, wherein each compartment comprises not more than a single microparticle.
Embodiment 170. The library of any one of embodiments 158 to 169, wherein the library is in a vessel.
Embodiment 171. The library of any one of embodiments 158 to 169, wherein each member of the library is in a vessel.
Embodiment 172. The library of any one of embodiments 158 to 171, wherein the target polypeptide is derived from a transcriptome.

### EXAMPLES

These examples are provided for illustrative purposes only and not to limit the scope of the claims provided herein.

### Example 1. Design and Use of a Spatially Encapsulated DNA Encoded Library

This example describes the design, preparation, and use of a spatially DNA encoded library.

Briefly, a set of large particles that are able to form particle template emulsions (i.e., 80 micron), are bifunctionally modified with a solution, comprised of 1% solution A (comprising an oligonucleotide) and 99% solution B, or varying % as required for the material. Solution A modifies the particle with an oligonucleotide that can capture primer A. The oligonucleotide has a cleavable base 5′ and at least 6 nt away from the base on the oligonucleotide that is forming the attachment with the particle. Solution B modifies the particle with an amino acid bearing a ‘clickable’ handle that can link an affinity tag of interest (i.e., SpyCatcher, or anti-GST antibody). The bifunctionalized particle is then incubated with cDNA template, or comparable, that can form a monoclonal copy of DNA on the particle after PCR, and vortexed in the presence of oil and surfactant to form particle templated emulsions for PCR with the cDNA template. It is estimated that roughly ~10M molecules can be attached at sites modified by Solution B, with ~100K sites for Solution A.

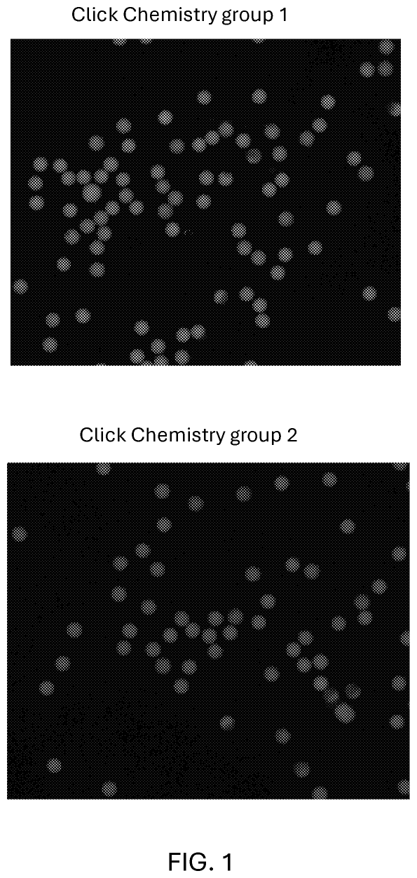

For the sites modified by Solution A, with an oligonucleotide-during the PCR amplification with a cDNA template, or comparable, in the presence of primers to amplify the cDNA, a copy of the cDNA is transferred to the particle. The primers include one primer that has a cleavage base at least 6 nt from the terminus of the 5′ end, such that when cleaved a 6 nt overhang is revealed after gentle denaturation and washing. After the cDNA is transferred, the particles contain dsDNA amplicon with a 5′ terminus bearing bases that can be cleaved to reveal 6 nt overhangs. The particles are then washed and isolated to reveal a population of particles with cDNA templates. Each cDNA template contains a region for encoding a protein of interest, a primer site 5′ to a protein barcode sequence, and a primer site 3′ of the protein barcode sequence (FIG. 1).

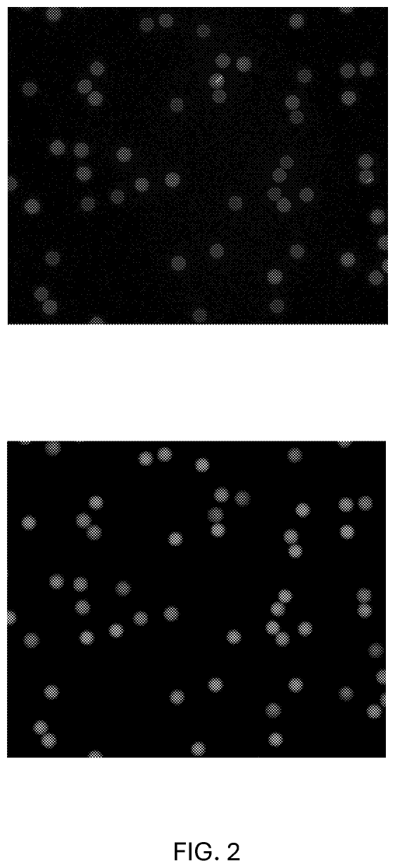

The isolated particles are then processed by cleaving the base at the 5′ terminus of the amplicon on the particles. For example, a uracil is cleaved with USER enzyme. The particles are washed with 10% DMSO/TE buffer, or comparable, to reveal ssDNA overhangs. The distal region from the particle, are processed such that split-and-pool ligation and short dsDNA oligonucleotides ligated at the distal end produces a ‘barcode’ sequence that identifies a single bead. The terminal ligation includes a dsDNA oligonucleotide that contains a nucleotide 6 nt from the 5′ end that can be cleaved to reveal a ssDNA overhang. Next, the amplicon is processed to reveal ssDNA overhangs at both ends of the amplicon, and then ligated with a ‘hairpin’ sequence to form a fully continuous closed sequence with no free 3′ OH region. This final construct is resistant to nuclease degradation in the presence of in vitro transcription translation lysate from cells (FIG. 2).

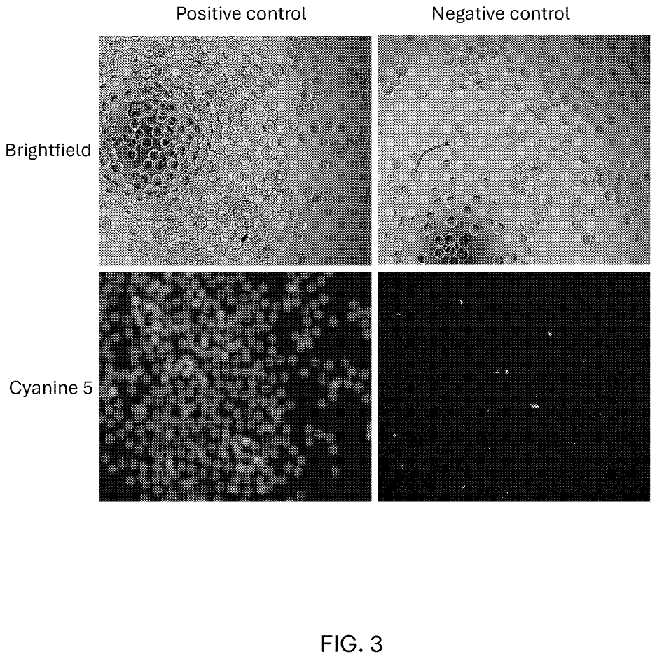

The processed clonal particles are then used for in vitro transcription translation (IVT) of a protein encoded by each particle's protein encoding sequence. The particles are incubated with IVT solution, on-ice, and then subjected to vortexing in the presence of oil/surfactant to encapsulate single particles in droplets. The temperature is then be raised to 30° C., or comparable, and protein is expressed within each droplet such that the protein contains a ‘tag’ that then binds to the affinity tag present on the particle introduced through Solution B. The reaction proceeds to completion such that all sites revealed from Solution B contains an attached protein. The particles are then be harvested upon droplet lysis, and washed to produce a library of particles containing both a protein encoding sequence, as well as the protein encoded by the sequence. Each particle is expected to contain ~10M proteins per particle, and ~100K dsDNA encoding sequences for the protein (FIG. 3).

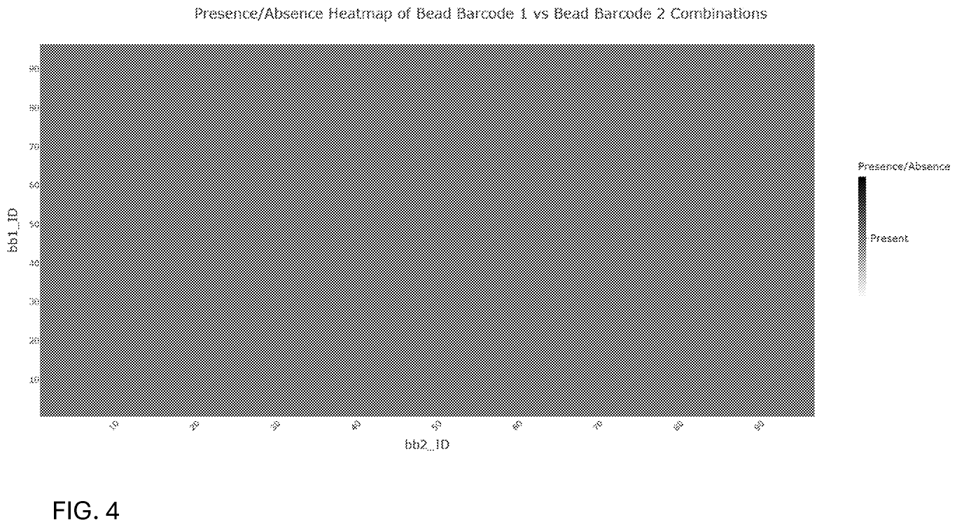

The population of particles containing the encoded protein sequence, and protein encoded by the sequence, is incubated with a solution of encoded molecules, for example, a DNA encoded library (DEL). The DEL is allowed to bind, and form either non-covalent or covalent interactions with the unique clonal proteins on each particle. Any unbound DEL is briefly washed away from the particle using conditions to remove false-positives, nonspecific interactions, or any nucleic acid mediated interactions with proteins on the particles (FIG. 4).

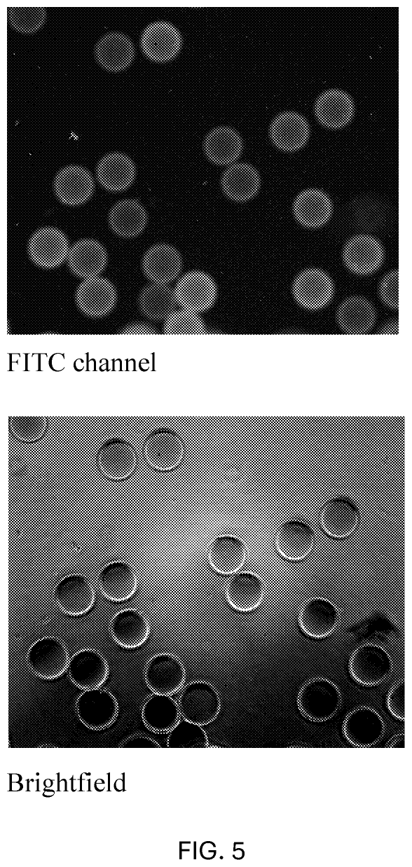

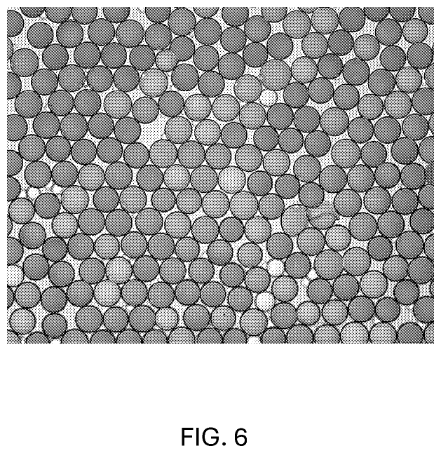

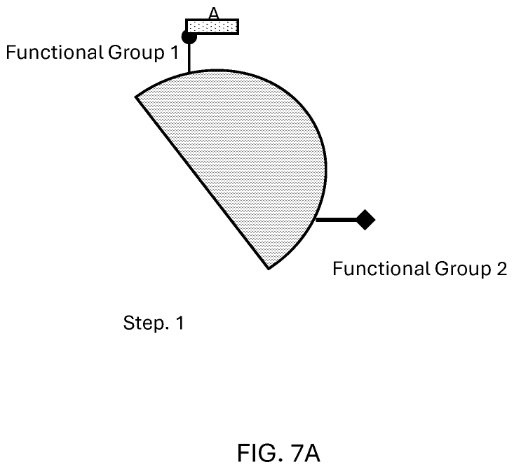

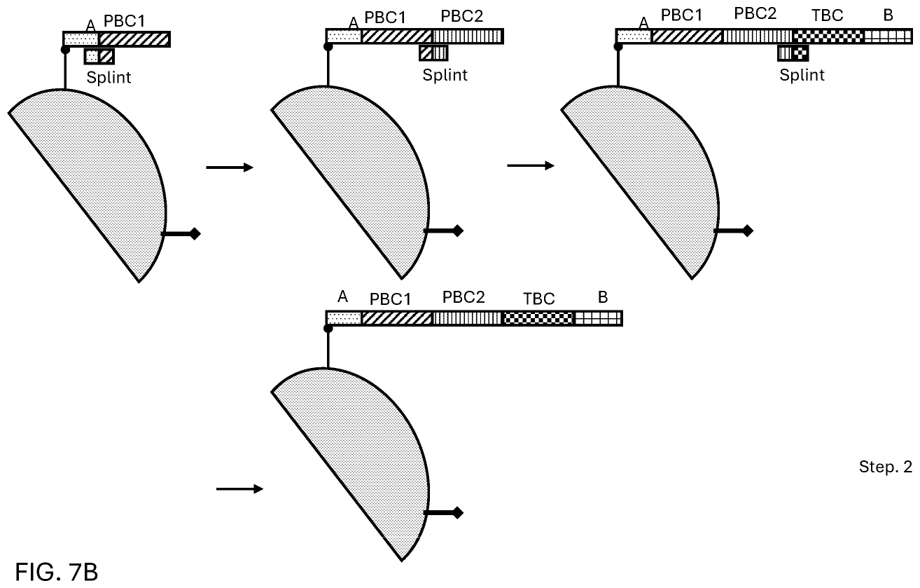

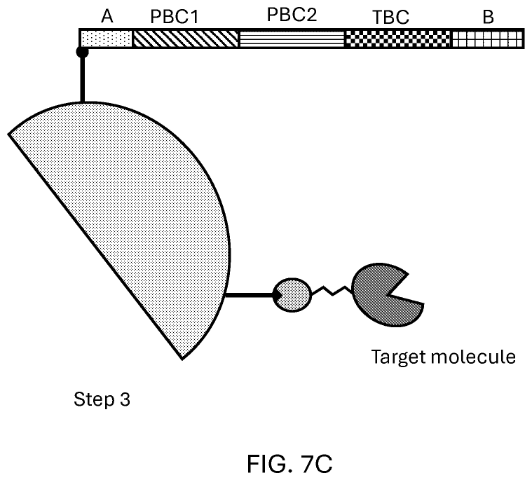

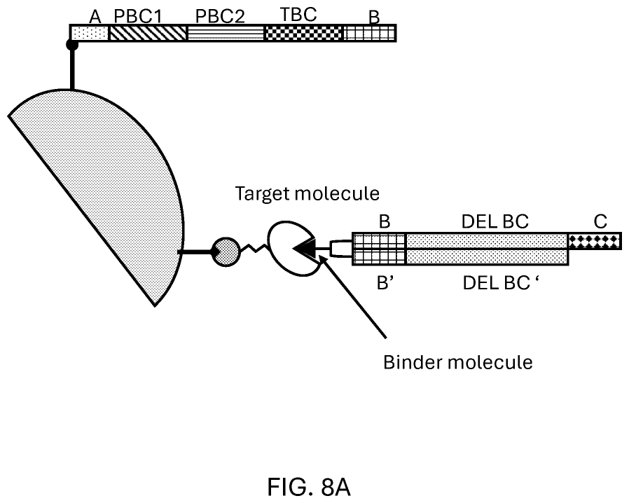

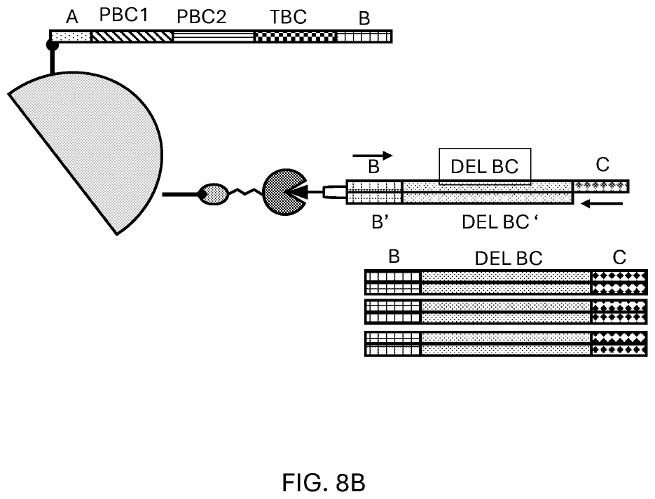

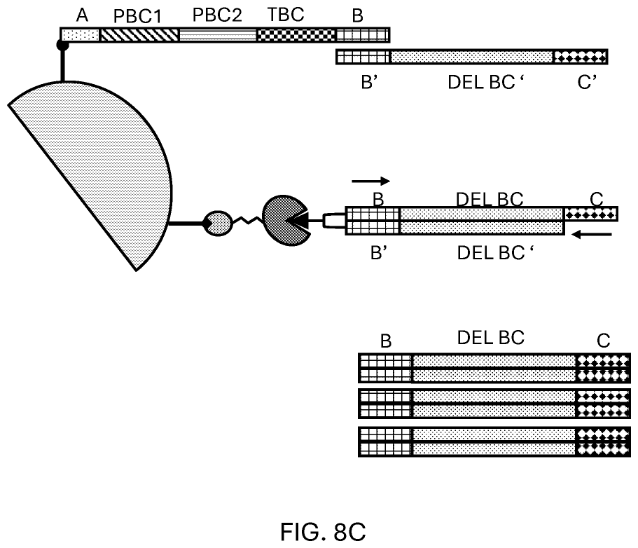

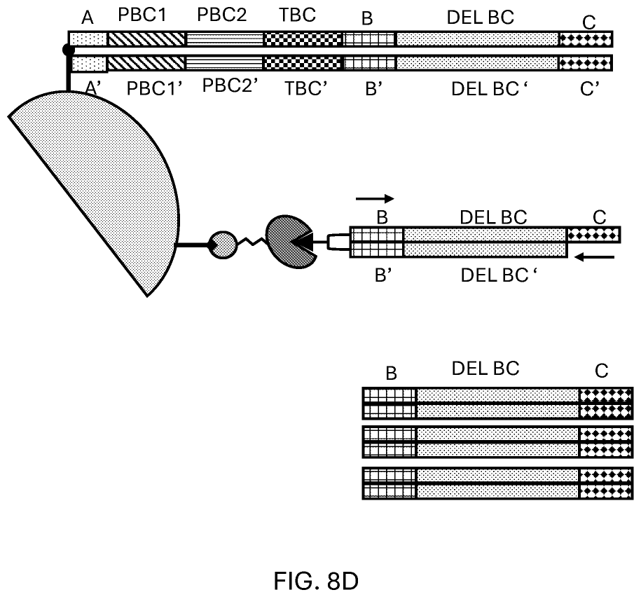

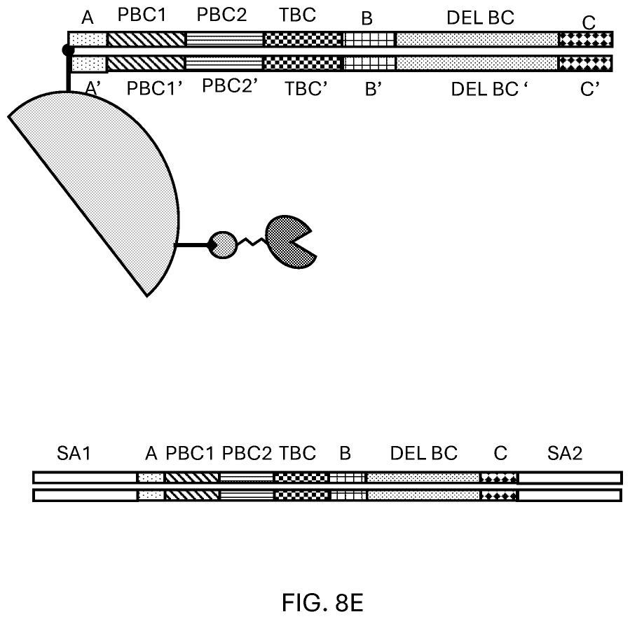

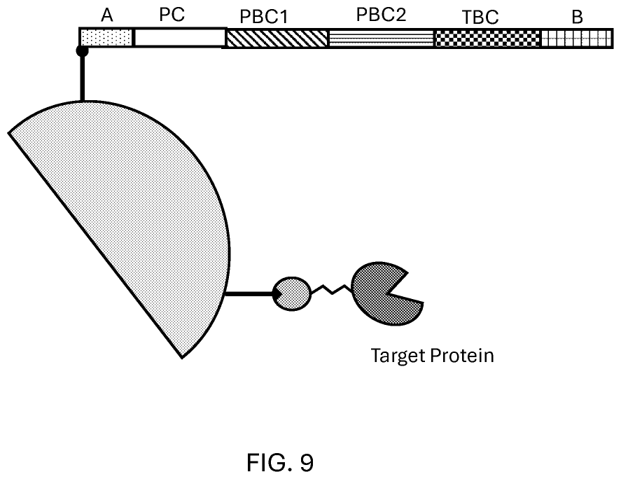

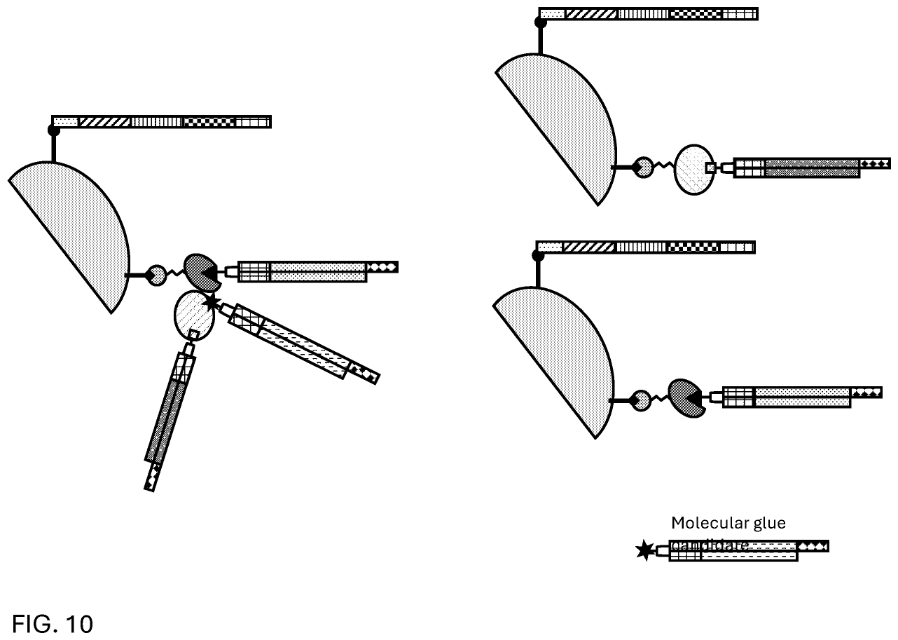

The population of particles containing encoded proteins, with covalently or non-covalently bounded DEL molecules, is then placed in solution containing PCR reagents with 2 sets of primers that amplifies the protein barcode and bead barcode region, as well as the DEL encoding region. The amplified products link together as there is complementary 3′ regions for each amplicon. The PCR reaction is initiated after the particles are encapsulated, such that one particle is in one emulsion, after vortexing the aqueous solution with oil/surfactant, and placed in a thermocycler. The full dsDNA amplicon products from the reaction contains the DEL encoding sequence, the protein encoding sequence, and the bead barcode sequence. The number of DEL members is counted that bound to a particular protein, by using a UMI for each amplicon, and the frequency of each DEL member that was bound to a protein is summed by determining the total number of unique beads it was present on with the same protein barcode sequence (FIG. 5).

### Example 2. Design and Use of a Spatially Encapsulated DNA Encoded Library for Discovery of Molecular Glues

This example describes the design, preparation, and use of a spatially DNA encoded library for the discovery of molecular glues, such as molecules that only bind when a pocket is revealed from the complex or interaction between two proteins.

Three screens are performed. In the first screen, one class of protein, for example an E3 ligase, is immobilized on particles, and subjected to a screen with a particular DEL (as described in Example 1). The results of DEL members that bound such E3's are recorded.

In the second screen, a target protein class, for example, a group of kinases or the entire proteome, is screened with a particular DEL. The results of DEL members that bound such a target class are recorded.

In the third screen, a specific E3 is incubated with a target protein class on particles, and a particular DEL are incubated with a library of complexed target proteins. The results of DEL members that bound these complexes, are recorded.

The DEL members that only bound that complex, but did not bind either the E3 ligase class or the target protein class only, reveal the molecules that only bind the protein complex tested (FIG. 6).

### Advantages of the Methods of the Disclosure

### Molecular Glue Discovery at Scale

Screen a protein library of protein modifiers (e.g., E3 or DUB) with a single target of interest (TOI) against billions of small molecules to identify which protein modifier can form a molecular glue with a TOI through chemically induced proximity.

### Extreme Selectivity

Screen a library of proteins from an entire class or pathway to find molecules that only bind the desired target. Leverage the fingerprint of selectivity to only pursue molecules that don't show undesired interactions with other proteins.

### Pocket Targeting

Screen a library of mutants of a protein target in order to disrupt binding. The goal is to determine what particular amino acid locations are necessary for molecular binding.

### Pharmacophore Mapping

Screen a library of mutants of a protein target in order to characterize binding interactions of a pocket (steric, donor, acceptor). The goal is to determine what particular amino acid residues/domains are necessary for molecular binding.

### Rapidly Launch Programs

Screen protein libraries, comprised of diverse protein surface and protein classes to construct foundational models by mapping protein structures and sequences to small molecule binding to predict what molecules will bind an arbitrary protein sequence.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 3. Preparation of Barcoded Protein Loaded Bifunctional Microparticles Microparticle Functionalization

Microparticles that can be used for screening the spatially encapsulated DNA encoded libraries are functionalized with two orthogonal click chemistry groups. One chemical group serves as the attachment point for an oligonucleotide (grafting primer), while the other group is designated for capture of a target molecule (e.g., an antibody for the capture of an affinity-tagged protein) (FIG. 1).

Briefly, microparticles were bi-functionalized via click chemistry. The efficiency of functionalization was confirmed by conjugating a Cyanine 5 dye via click chemistry. The results show, that 99.9% of the microparticles were successful labeled. The efficiency of the click chemistry reaction for attaching the grafting primer was validated by the hybridization of a Cy5-labeled complementary probe, while the attachment of the antibody was confirmed by binding a cGST-tagged GFP protein (FIG. 2).

### Microparticle Barcoding

Microparticles used for DNA-Encoded Library (DEL) selection were barcoded through sequential DNA ligations. This process involved the splint ligation of single-stranded DNA oligonucleotide using T4 DNA ligase. To encode the microparticles with unique particle identifiers, a split-and-pool ligation strategy was employed. Two or more DNA modules were ligated in a 96-well plate format, with each module comprising 96 different sequences. This method allows for a potential barcode diversity of 96N, where N represents the number of DNA modules used. The use of two or more DNA molecules to prepare a combined unique barcode is advantageous, since single DNA barcode module are impractical due to the need for a large number of unique DNA sequences.

After ligating the final particle-encoding module, the microparticles were pooled and split a final time to ligate a unique barcode identifier specific to the protein target to be attached to the particle. The success of each ligation was monitored using labeled DNA probes that hybridize the ligated DNA module (FIG. 3). The quality of the completed microparticle particle barcodes was also assayed through quantitative PCR (qPCR) and Next Generation Sequencing (NGS) (FIG. 4).

The success of each ligation was monitored using labeled DNA probes that hybridize the ligated DNA module. A successful staining of 99% of the microparticles with the labeled probe was considered a passing quality control criterion. The quality of the completed microparticle particle barcodes was tested by quantitative PCR (qPCR) and the diversity of the particle barcode combinations was tested by Next Generation Sequencing (NGS). These tests show that microparticle barcoding were successful in all the samples tested and that all combination of microparticle barcodes were present.

### Protein Immobilization

Recombinant C terminal GST tagged target proteins were incubated for 2 hours at room temperature with the functionalized microparticles in a physiological buffer. The attachment of the proteins was assayed with a FITC labeled anti GST antibody (FIG. 5). The staining shows that more than 90% of the microparticles were successfully labeled with target protein.

### Emulsion PCR

During this PCR, the microparticle DNA barcode is linked to the DNA of the DNA encoded chemical molecule to form a concatenated amplicon that links the information of the target protein immobilized on the microparticle and the DEL molecule binders. This step also adds next generation sequencing adapters. Post selection microparticles were collected and then incubated with a PCR mix for 5 minutes at room temperature to allow for the PCR reagents to diffuse into the particles. Particle templated emulsion PCR is described in the art, for example in Hatori et al., Analytical Chemistry (2018) 90, 9813-9820.

After incubation, all the supernatant was removed and 2 to 5% of fluorosurfactant in a fluorinated oil was added before vortexing the mixture to generate emulsion droplets. The emulsified microparticles were then transferred in a PCR tube for thermocycling.

Post PCR, microparticles and amplicons were recovered by breaking the emulsion with 20% (v/v) perfluoro-1-octanol in fluorinated oil to separate the aqueous and oil phase. Microparticles and PCR products in the aqueous phase were collected for downstream NGS library preparation. Expected sizes for PCR products are shown in Table 1.

TABLE 1
Emulsion PCR products sizes

| | Size | |
|---|---|---|
| | (base | |
| Emulsion PCR products | pairs) | |
| Concatenated microparticle and DEL | 290 | Final product |
| Microparticle barcode | 150 | Intermediary product |
| DNA encoded chemical library | 159 | Intermediary product |

### NGS Sample Preparation

Purified amplicons from the emulsion PCR were indexed during a second PCR using Illumina indexes. Illumina libraries were then purified and quantified before sequencing. See expected sizes for PCR products are shown in Table 2.

TABLE 2
Indexing PCR products.

| Indexing PCR product | Size (base pairs) |
|---|---|
| Indexed emPCR product | 329 |

### Example 4. DNA Encoded Library Selection (DEL) Using Bifunctional Microparticles

The bifunctional microparticles can be used for screening the spatially encapsulated DNA encoded libraries.

Briefly, bifunctional microparticles can be functionalized with proteins of choice, for example protein kinases. Seventeen protein targets (Table 3) were immobilized on barcoded bifunctional microparticles as described in Example 3.

After the target proteins were immobilized, microparticles harboring multiple target proteins were pooled together and incubated with a DNA encoded chemical library (DEL) for 1 to 2 hours at room temperature under constant agitation. Unbound DEL molecules were washed away using multiple rounds of selection buffer washes. Microparticles with selected compounds were then carried through an emulsion PCR.

Unlike a classical DEL selection, the barcoding of the microparticle and degenerate sequences in the DEL barcode allow for the counting of the number of DEL molecules selected and the number of microparticles associated with each binding event.

Seventeen protein targets immobilized on barcoded microparticles and an empty microparticle control were used in a DEL screen with twenty-five-million-member DEL library spiked with a control compound X known to bind to the BRD4 protein. Table 3 summarizes the counts of binding events of compound X to each of the protein target and control. The data shows that compound X is highly enriched on beads harboring the BRD4 proteins.

TABLE 3
Multiplexed DEL selection

| | Number of | Number of Compound |
|---|---|---|
| Target Protein | microparticles | X molecules selected |
| BRD4 | 85 | 187 |
| Truncated BRD4 | 6 | 11 |
| p38gamma | 5 | 7 |
| JAK1 | 5 | 7 |
| p38delta | 5 | 7 |
| p38beta | 5 | 6 |
| PDE1a | 4 | 4 |
| YES1 | 4 | 4 |
| AURKA | 4 | 4 |
| JAK2 | 3 | 4 |
| HER2 | 4 | 4 |
| JAK2 V617F | 3 | 3 |
| BTK_1_659 | 3 | 3 |
| MAPK14 | 2 | 2 |
| Empty microparticles | 5 | 5 |
| TYK2 | 1 | 1 |
| FGFR3 (CD333) | 1 | 1 |
| ROR1 | 1 | 1 |

Similar results were obtained with a classical single plex DEL selection for BRD4 binders, where a 60 fold enrichment of the BRD4 control compound X with BRD4 protein against the control protein MAPK14.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

What is claimed is:

1. A method of screening for one or more DNA encoded compounds that bind to a target polypeptide, the method comprising:
(i) contacting a plurality of DNA encoded compounds with a target polypeptide bound to a microparticle under conditions that allow for binding of the plurality of DNA encoded compounds to the target polypeptide, wherein the microparticle is bound to a nucleic acid multifunctional sequence, wherein said nucleic acid multifunctional sequence comprises:
a nucleic acid polypeptide identifier sequence; and
a first hybridization sequence;
wherein each DNA encoded compound of the plurality of DNA encoded compounds comprises a compound bound to a DNA compound sequence,
wherein the DNA compound sequence comprises:
a nucleic acid compound identifier sequence; and
a second hybridization sequence wherein a complement of the second hybridization sequence is capable of hybridizing to said first hybridization sequence,
(ii) removing DNA encoded compounds not binding to the target polypeptide;
(iii) compartmentalizing each microparticle;
(iv) amplifying the DNA compound sequence to produce a complement of the DNA compound sequence, wherein the complement of the DNA compound sequence comprises a complement of the second hybridization sequence;
(v) hybridizing the complement of the second hybridization sequence to the first hybridization sequence; and
(vi) elongating the nucleic acid multifunctional sequence with a polymerase to produce a nucleic acid identification sequence comprising the nucleic acid compound identifier sequence and the nucleic acid polypeptide identifier sequence,
thereby identifying the one or more DNA encoded compounds that bind to a target polypeptide. A method of screening for one or more DNA encoded compounds that bind to a target polypeptide, the method comprising:
(i) contacting a plurality of DNA encoded compounds with a plurality of target polypeptides bound to a microparticle under conditions that allow for binding of the plurality of DNA encoded compounds to the polypeptides, wherein the plurality of target polypeptides bound to a microparticle comprises a nucleic acid multifunctional sequence bound to said microparticle, wherein said nucleic acid multifunctional sequence comprises:
a nucleic acid polypeptide identifier sequence; and
a first hybridization sequence;
wherein a DNA encoded compound of the plurality of DNA encoded compounds comprises a compound bound to a DNA compound sequence,
wherein the DNA compound sequence comprises:
a nucleic acid compound identifier sequence; and
a second hybridization sequence wherein the complement of the second hybridization sequence is capable of hybridizing to said first hybridization sequence,
(ii) removing DNA encoded compounds not binding to a target polypeptide;
(iii) compartmentalizing each microparticle; and
(iv) amplifying the DNA compound sequence to produce a complement of the DNA compound sequence, wherein the complement of the DNA compound sequence comprises a complement of the second hybridization sequence;
(v) hybridizing the complement of the second hybridization sequence to the first hybridization sequence; and
(vi) elongating the nucleic acid multifunctional sequence with a polymerase to produce a nucleic acid identification sequence comprising the nucleic acid compound identifier sequence and the nucleic acid polypeptide identifier, thereby identifying the one or more DNA encoded compounds that bind to a target polypeptide. The method of claim 1, wherein the method further comprises de-coding the compound from the corresponding nucleic acid compound identifier sequence.

2. The method of claim 1, wherein the method comprises a plurality of target polypeptides.

3. The method of claim 4, wherein the target polypeptides are the same target polypeptide.

4. The method of claim 4, wherein the target polypeptides are different target polypeptides.

5. The method of claim 1, wherein the DNA encoded compounds are the same compound.

6. The method of claim 1, wherein the DNA encoded compounds are different compounds.

7. The method of claim 1, wherein the microparticles are compartmentalized in an emulsion.

8. The method of claim 1, wherein the microparticles are compartmentalized in a vessel.

9. The method of claim 1, the method further comprising identifying the one or more target polypeptides that specifically bind to a DNA encoded compound by de-coding from corresponding nucleic acid polypeptide identifier nucleic acid sequences.

10. The method of claim 1, further comprising amplifying the nucleic acid identification sequence comprising the nucleic acid polypeptide identifier sequence, the first hybridization sequence, and the nucleic acid compound identifier sequence.

11. The method of claim 12, further comprising sequencing the nucleic acid identification sequence.

12. The method of claim 1, further comprising identifying and determining unique identifier sequencing events.

13. The method of claim 14, further comprising determining the number of target polypeptide binding events for each unique nucleic acid compound identifier sequence.

14. The method of claim 15, further comprising determining a target polypeptide binding frequency for each DNA encoded compound of the plurality of DNA encoded compounds.

15. The method of claim 16, further comprising summing all target polypeptide binding events across two or more microparticles comprising the same target polypeptide.

16. The method of claim 17, further comprising determining a distribution of target polypeptide binding events.

17. The method of claim 1, wherein the method steps are repeated.

18. The method of claim 19, further comprising determining a distribution of target polypeptide binding events over 2 or more repeats.

19. A computer-implemented method for identifying a compound having an affinity with a target polypeptide, the method comprising
querying a machine learning engine for a proposed compound having an affinity with the target polypeptide, wherein the machine learning engine was trained using the target polypeptide binding events of claim 1; and
receiving the compound from the machine learning engine.

20. A composition comprising
(i) a microparticle;
(ii) a target molecule bound to said microparticle; and
(iii) a nucleic acid multifunctional sequence bound to said microparticle, wherein said nucleic acid multifunctional sequence comprises:
a nucleic acid target molecule identifier sequence; and
a first hybridization sequence.

21. A composition comprising
(i) a microparticle;
(ii) a target molecule bound to said microparticle;
(iii) a nucleic acid multifunctional sequence bound to said microparticle, wherein said nucleic acid multifunctional sequence comprises:
a nucleic acid target molecule identifier sequence; and
a first hybridization sequence; and
(iv) a DNA encoded compound bound to said target molecule, said DNA encoded compound comprising a compound bound to a DNA compound sequence, said DNA compound sequence comprising:
a nucleic acid compound identifier sequence; and
a second hybridization sequence wherein a complement of the second hybridization sequence is capable of hybridizing to said first hybridization sequence.

22. A composition comprising
(i) a microparticle;
(ii) a target molecule bound to said microparticle;
(iii) a nucleic acid multifunctional sequence bound to said microparticle, wherein said nucleic acid multifunctional sequence comprises:
a nucleic acid target molecule identifier sequence;
a first hybridization sequence; and
(iv) a DNA encoded compound bound to said nucleic acid multifunctional sequence, said DNA encoded compound comprising a compound bound to a DNA compound sequence, said DNA compound sequence comprising:
a nucleic acid compound identifier sequence; and
a complement of a second hybridization sequence, wherein the complement of the second hybridization sequence and the first hybridization sequence are bound to each other.

23. The composition of claim 22, wherein the target molecule is a target polypeptide.

24. A composition comprising
(i) a microparticle;
(ii) a target polypeptide bound to said microparticle;
(iii) a nucleic acid multifunctional sequence bound to said microparticle, wherein said nucleic acid multifunctional sequence comprises:
a nucleic acid polypeptide identifier sequence; and
a first hybridization sequence; and
(iv) a DNA encoded compound bound to said target polypeptide, said DNA encoded compound comprising a compound bound to a DNA compound sequence, said DNA compound sequence comprising:
a nucleic acid compound identifier sequence; and
a second hybridization sequence wherein a complement of the second hybridization sequence is capable of hybridizing to said first hybridization sequence.

25. A composition comprising
(i) a microparticle;
(ii) a target polypeptide bound to said microparticle;
(iii) a nucleic acid multifunctional sequence bound to said microparticle, wherein said nucleic acid multifunctional sequence comprises:
a nucleic acid polypeptide identifier sequence;
a first hybridization sequence; and
(iv) a DNA encoded compound bound to said nucleic acid multifunctional sequence, said DNA encoded compound comprising a compound bound to a DNA compound sequence, said DNA compound sequence comprising:
a nucleic acid compound identifier sequence; and
a complement of a second hybridization sequence, wherein the complement of the second hybridization sequence and the first hybridization sequence are bound to each other.

26. The composition of claim 22, wherein the nucleic acid multifunctional sequence further comprises a target polypeptide encoding nucleic acid sequence.

27. The composition of claim 22, further comprising a nucleic acid identification sequence comprising the nucleic acid target molecule identifier sequence or nucleic acid polypeptide identifier sequence, the first hybridization sequence, and the nucleic acid compound identifier sequence.

28. The composition of claim 22, wherein the microparticle is a hydrogel.

29. The composition of claim 22, wherein the longest dimension of the microparticle is at least 1 μm.

30. The composition of claim 22, wherein the longest dimension of the microparticle is at most 500 μm.

31. The composition of claim 22, wherein the microparticle is a bead.

32. The composition of claim 22, wherein the target molecule further comprises a protein tag.

33. The composition of claim 34, wherein the protein tag is selected from the group of Polyhistidine (His-tag), Glutathione S-transferase (GST), FLAG tag, SPY tag, calmodulin binding domain (CBD), chitin binding domain (CBD), choline-binding domain (CBD), albumin-binding protein (ABP), Bacteriophage T7 epitope (T7-tag), bacteriophage V5 epitope (V5-tag), biotin-carboxy carrier protein (BCCP), bluetongue virus tag (B-tag), chloramphenicol acetyl transferase (CAT), cellulose binding domain (CBP), E2 epitope, galactose-binding protein (GBP), green fluorescent protein (GFP), Glu-Glu (EE-tag), human influenza hemagglutinin (HA), HaloTag, maltose-binding protein (MBP), streptavadin-binding peptide (SBP), Myc epitope tag, strep-tag, and biotin.

34. The composition of claim 22, wherein the target molecule is bound non-covalently to the microparticle.

35. The composition of claim 22, wherein the target molecule is bound covalently to the microparticle.

36. The composition of claim 22, wherein the target molecule is bound to the microparticle by a target polypeptide binding moiety.

37. The composition of claim 38, wherein the target molecule binding moiety is selected from the group of a divalent metal, glutathione, anti-FLAG antibody or fragment thereof, calmodulin, chitin, choline, anti-E2 antibody or a fragment thereof, cellulose, albumin, streptavidin, anti-bluetongue virus antibody or a fragment thereof, galactose, anti-GFP antibody or a fragment thereof, biotin, polypeptide specific antibody, and maltose.

38. The composition of claim 38, wherein the target polypeptide binding moiety further comprises a linker.

39. The composition of claim 40, wherein the linker is a polypeptide linker, a chemical linker, or a nucleic acid linker.

40. The composition of claim 22, wherein the composition is encapsulated.

41. The composition of claim 22, wherein the composition is partitioned into a compartment.

42. The composition of claim 43, wherein the compartment is an aqueous droplet within an emulsion.

43. The composition of claim 44, wherein the emulsion is an oil in water emulsion.

44. The composition of claim 43, wherein the longest dimension of the compartment is between 10 μm and 1 mm.

45. The composition of claim 22, wherein the compound is selected from the group of a small molecule, a macromolecule, a peptide, a polypeptide, a protein, a protein complex, a nucleic acid, a lipid, a carbohydrate, a glycan, and a cell.

46. The composition of claim 47, wherein the compound is a protein complex.

47. The composition of claim 47, wherein the compound is a neoantigen peptide.

48. The composition of claim 47, wherein the compound is a RNA, a DNA, or a mixture of RNA or DNA.

49. The composition of claim 47, wherein the compound is an aptamer.

50. The composition of claim 22, wherein the nucleic acid multifunctional sequence is bound to the microparticle by a nucleic acid linker or a chemical linker.

51. The composition of claim 22, wherein the nucleic acid multifunctional sequence further comprises a unique identifier.

52. The composition of claim 22, wherein the nucleic acid multifunctional sequence is a DNA, RNA, or a mixture of DNA and RNA.

53. The composition of claim 22, wherein the nucleic acid multifunctional sequence is a single stranded nucleic acid.

54. The composition of claim 22, wherein the nucleic acid multifunctional sequence further comprises a first amplification sequence.

55. The composition of claim 23, wherein the DNA compound sequence further comprises a second amplification sequence.

56. The composition of claim 22, wherein the nucleic acid multifunctional sequence is resistant to nuclease degradation.

57. The composition of claim 22, wherein about 10⁵ to 10⁶ of said nucleic acid multifunctional sequences are attached to the microparticle.

58. The composition of claim 22, wherein about 10⁶ to 10⁹ of said target molecules are attached to the microparticle.

59. A method of making a composition of claim 22, the method comprising combining the microparticle, a target polypeptide binding moiety, the target polypeptide, the nucleic acid multifunctional sequence under conditions sufficient for (i) attaching the target polypeptide binding moiety and the nucleic acid multifunctional sequence to the microparticle, and (ii) binding the target polypeptide to the target polypeptide binding moiety.

60. A method of making a composition of claim 22, the method comprising combining the microparticle, a target polypeptide binding moiety, the target polypeptide, the nucleic acid multifunctional sequence and the DNA encoded compound under conditions sufficient for (i) attaching the target polypeptide binding moiety and the nucleic acid multifunctional sequence to the microparticle, (ii) binding the target polypeptide to the target polypeptide binding moiety; and (iii) binding the DNA encoded compound to the target polypeptide.

61. The method of claim 61, wherein step (i) comprises covalently conjugating the multifunctional sequence to the microparticle.

62. The method of claim 61, wherein step (i) comprises covalently conjugating a target polypeptide binding moiety to the microparticle.

63. The method of claim 61, wherein the conjugation is a chemical conjugation.

64. The method of claim 61, wherein the microparticle comprises one or more functional reactive groups.

65. The method of claim 66, wherein the functional reactive group is an amine group or, a carboxy group.

66. The method of claim 67, wherein the functional reactive group is functionalized with a dibenzylcyclooctyne (DBCO).

67. The method of claim 68, further comprising, reacting the DBCO with a nucleic acid multifunctional sequence azide.

68. The method of claim 69, further comprising, reacting the DBCO with a target polypeptide binding moiety azide.

69. The method of claim 61, further comprising encapsulating the composition.

70. The method of claim 61, further comprising producing the target polypeptide in situ by transcribing and translating the polypeptide encoding nucleic acid sequence.

71. A library, wherein the library comprises a plurality of compositions of claim 22, or a plurality of compositions produced by the method of claim 62.

72. The library of claim 73, wherein the library comprises a plurality of target polypeptides.

73. The library of claim 74, wherein the target polypeptides are the same target polypeptide.

74. The library of claim 75, wherein the target polypeptides are different target polypeptides.

75. The library of claim 73, wherein the library comprises a plurality of DNA encoded compounds.

76. The library of claim 77, wherein the DNA encoded compounds are the same compound.

77. The library of claim 77, wherein the DNA encoded compounds are different compounds.

78. The library of claim 73, wherein the library is in an emulsion.

79. The library of claim 73, wherein each member of the library is encapsulated.

80. The library of claim 73, wherein each member of the library is partitioned into a different compartment.

81. The library of claim 82, wherein each compartment is an aqueous droplet in an emulsion.

82. The library of claim 83, wherein the emulsion is an oil in water emulsion.

83. The library of claim 82, wherein each compartment comprises not more than a single microparticle.

84. The library of claim 73, wherein the library is in a vessel.

85. The library of claim 73, wherein each member of the library is in a vessel.

86. The library of claim 73, wherein the target polypeptide is derived from a transcriptome.
